(19) United States
(12) Patent Application Publication
(10) Pub. No.: US 2025/0085237 A1
(43) Pub. Date: Mar. 13, 2025

(54) RADIOFREQUENCY SENSOR SYSTEM

(71) Applicant: LIFE TECHNOLOGIES CORPORATION, Carlsbad, CA (US)

(72) Inventors: Mark Thomas Smith, Nibley, UT (US); Ronald Abraham Martin, Smithfield, UT (US); Brycen Hal Mills, Millville, UT (US); Joshua Lynn Adams, Smithfield, UT (US); Jia Zeng, North Logan, UT (US); Riley Baranowski, Somerville, MA (US)

(21) Appl. No.: 18/882,568

(22) Filed: Sep. 11, 2024

Related U.S. Application Data
(60) Provisional application No. 63/582,486, filed on Sep. 13, 2023.

(51) Int. Cl. G01N 22/00; C12M 1/34; C12M 3/00

(52) U.S. Cl. CPC G01N 22/00; C12M 23/46; C12M 41/32

(57) ABSTRACT

Devices, systems, and methods for measuring an analyte are disclosed. A device, system, or method can include a radiofrequency sensor comprising a radiofrequency transmitter and a radiofrequency receiver. A device, system, or method can be configured to transmit a radiofrequency signal from the transmitter to the receiver through a culture medium comprising the analyte. In some embodiments, a device, system, or method can include a controller configured to determine a concentration of the analyte based on a resultant signal produced at the receiver. A device, system, or method can comprise a radiofrequency sensor introducer. An introducer of a device, system, or method can comprise a first sensor sheath configured to receive a radiofrequency transmitter and a second sensor sheath configured to receive a radiofrequency receiver. In some implementations, an introducer of a device, system, or method can be configured to maintain a separation between the transmitter and receiver.

### CROSS-REFERENCE TO RELATED APPLICATIONS

This application claims the benefit of the U.S. Provisional Patent Application Ser. No. 63/582,486 filed Sep. 13, 2023, and titled “RADIOFREQUENCY SENSOR SYSTEM,” which is incorporated herein by specific reference.

### BACKGROUND

Bioreactor systems are frequently used in the biopharmaceutical industry for a variety of processes such as in the preparation of media, buffers, and biologics (such as cytokines and antibodies) and in the growing, mixing and/or suspension of mammalian cells and microorganisms (e.g., for the production of media, buffers, and/or biologics). Cell culture conditions within bioreactor systems can be critical to the quality of the product(s) produced with the bioreactor systems and/or to the efficiency of the production process. Accordingly, monitoring and maintenance of bioprocess conditions in bioreactors can be critical to the quality and efficiency of bioreactor production processes.

Bioprocessing within bioreactors has traditionally relied on a variety of measurement methodologies, namely in-line, on-line, at-line, and offline. The offline approach, while once prevalent, involves extracting samples from the bioreactor for the assessment using external instruments or shipping them to third-party laboratories for further analysis. However, this technique presents certain limitations. For example, it lacks real-time fidelity to the conditions within the bioreactor, which can result in bias and delays when adjustments or analyses are needed. Additionally, the sample extraction process may inadvertently introduce contaminants, potentially jeopardizing the entire run. In contrast, the other measurement methods offer distinct advantages. On-line measurements involve sensors outside the bioreactor that analyze the process parameters in real time, offering a greater degree of immediacy compared to offline methods. At-line measurements entail taking a sample from the bioreactor and analyzing it near the production line, allowing for more prompt feedback than offline methods. However, the in-line measurement garners attention due to advancements in Process Analytical Technology (PAT). In-line methods directly incorporate probe-like sensors into the bioreactors, enabling real-time, continuous bioprocess monitoring. This yields data of higher integrity and enhances overall operational efficacy by offering immediate insights into the bioreactor's status.

Cell culture conditions in bioreactors are often measured by sampling cell culture medium as a bioreactor process is performed, using current technologies. Such techniques allow accurate determination of certain cell culture conditions; however, there can be several drawbacks with such approaches. Sampling cell culture medium can require temporarily halting mixing processes, which can, in some situations, cause transient changes in delicate bioreactor process conditions. In some cases, a sterile environment of a bioreactor culture chamber must be opened to an outside environment or even contacted with a sampling cup introduced from outside of the bioreactor system, which can introduce risk for contamination of the bioreactor. An autosampler can be used to obtain samples of the cell culture medium for analysis; however, these systems can be expensive to purchase and adapt to a bioreactor system. Current autosampler technology can be limited in deployment to multiple-biorcactor or high-throughput configurations. Furthermore, current autosampler technologies cannot provide real-time measurement data, instead being limited to sampling rates of one sample every few hours. Directly sampling cell culture medium from a bioreactor to determine cell culture conditions can also add complexity and cost to a bioreactor system, as samples must still be analyzed on additional, potentially expensive equipment (e.g., mass spectrometry systems). Such workflows can quickly result in process bottlenecks, as it may be time intensive to analyze samples from multiple bioreactors in parallel on a limited number of analyzers, or in prohibitive cost barriers and decreased efficiency, if an effort is made to purchase more analyzers.

Over recent years, both Raman spectroscopy and Fourier-Transform Infrared (FTIR) spectroscopy have risen to prominence as promising in-line Process Analytical Technology (PAT) tools. As vibrational spectroscopic techniques, they are capable of providing extensive molecular information about the liquid solution in bioreactors. Their non-invasive nature further maintains the integrity of samples, preserving their quality. When incorporated into a probe, these technologies can continually monitor and detect contents within a bioreactor in real time. Moreover, combined with multivariate analysis tools, they extract more valuable information from complex systems. Despite their potential, these technologies present several limitations. Foremost among these is cost, largely attributed to the cost of laser emission and transmission devices integral to these spectroscopic techniques and the cost to maintain the systems. Additionally, probe longevity is a concern due to a limit on autoclave cycles and sterility is essential when contacting cell culture. Sensitivity also poses a challenge, especially for less concentrated solutes within the bioreactor system. Enhancing signals may necessitate additional cost and effort. Lastly, vibrational spectroscopy is affected by various factors, including an aqueous environment, fluorescence, ambient light, and variations in optic fibers. Hence, if there exists an alternative for a cost-effective, non-invasive (and even non-contact) device with improved performance, such a development could provide a significant advancement in the field of in-line PAT tools for bioprocessing.

### SUMMARY

In a first aspect of the present disclosure, a system for measuring an analyte is provided. The system includes a radiofrequency sensor comprising a radiofrequency transmitter and a radiofrequency receiver, and the sensor configured to transmit a radiofrequency signal from the transmitter to the receiver through a fluid comprising the analyte. The system further includes a controller comprising a processor and a non-transitory memory in communication with the sensor, the non-transitory memory comprising instructions that, when executed, cause the processor to determine a concentration of the analyte based on a resultant signal produced by the receiver.

In another aspect of the present disclosure, a method for measuring an analyte is provided. The method includes transmitting a radiofrequency signal from a radiofrequency transmitter into a fluid comprising an analyte; receiving a resultant radiofrequency signal at a radiofrequency receiver, wherein the resultant radiofrequency signal is the transmitted radiofrequency signal after it has passed through at least a portion of fluid; and analyzing the radiofrequency signal received by the receiver to determine a concentration of the analyte in the fluid.

In another aspect of the present disclosure, a device for measuring an analyte is provided. The device includes a radiofrequency sensor comprising a radiofrequency transmitter and a radiofrequency receiver; and an introducer comprising: the radiofrequency sensor and a coupling interface configured to couple the introducer to a container comprising the analyte.

In another aspect of the present disclosure, a system for measuring an analyte in a fluid contained in a bioprocessing container is provided. The system includes a housing, configured to receive the bioprocessing container. Further, the system consists of a first radiofrequency antenna holder and a second radiofrequency antenna holder, coupled to a top and bottom portion, respectively, of the housing. The first radiofrequency antenna holder consists of a first radiofrequency antenna, and the second radiofrequency antenna holder consists of a second radiofrequency antenna. The first and second radiofrequency antennas are configured to measure the analyte in the fluid.

In another aspect of the present disclosure, a system for measuring an analyte in a fluid contained in a bioreactor is provided. The system includes a radiofrequency probe and a radiofrequency holder coupled to the radiofrequency probe. The radiofrequency holder is configured to be received in a port of the bioreactor so that the radiofrequency probe is received inside the bioreactor.

It is understood that each aspect of the present disclosure herein can include any of the features, options, systems components, method steps, and possibilities recited in association with the any other aspect of the present disclosure or other embodiments set forth above or elsewhere within this application.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIGS. 1A-1H illustrate embodiments of a radiofrequency sensor system, in accordance with embodiments;

FIGS. 2A and 2B illustrate embodiments of radiofrequency sensors, in accordance with embodiments;

FIG. 2C shows a schematic of a radiofrequency sensor system, in accordance with embodiments;

FIGS. 3A-3C illustrate embodiments of radiofrequency sensor systems comprising a housing, in accordance with embodiments;

FIG. 4 illustrates a radiofrequency sensor system comprising a housing having a plurality of cavities, in accordance with embodiments;

FIGS. 5A-5D illustrate resultant signals produced by a radiofrequency sensor system, in accordance with embodiments;

FIGS. 6-8 show schematics of radiofrequency sensor systems, in accordance with embodiments;

FIGS. 9A and 9B illustrate an introducer, in accordance with embodiments;

FIG. 10 illustrates an introducer, a radiofrequency sensor, and a port adapter of a radiofrequency sensor system, in accordance with embodiments;

FIG. 11 shows components of a radiofrequency sensor system, including an introducer and a port adapter, in accordance with embodiments;

FIG. 12 shows components of a radiofrequency sensor system, including an introducer and a port adapter, in accordance with embodiments;

FIG. 13 shows components of a radiofrequency sensor system, including an introducer and a port adapter;

FIG. 14 shows a schematic of a controller of a radiofrequency sensor system, in accordance with embodiments;

FIGS. 15-17 show diagrams of a method for measuring one or more analytes of interest in a radiofrequency sensor system, in accordance with embodiments;

FIGS. 18 and 19 show radiofrequency (RF) predicted versus reference glucose values for glucose concentrations in fresh media (FIG. 18) or in spent cell culture media (FIG. 19), in accordance with embodiments;

FIGS. 20A-20B and 21A-21B show RF predicted versus reference glucose lactate mixtures, in accordance with embodiments;

FIG. 22 shows radiofrequency (RF) mean cross-validation R2 values for predicted versus reference measurements for detected amino acids in water, in accordance with embodiments;

FIGS. 23-29 show radiofrequency (RF) predicted versus reference concentrations of different amino acids in water, in accordance with embodiments;

FIG. 30A and FIG. 30B show radiofrequency (RF) predicted versus reference concentrations for a glutamine arginine mixture in water, in accordance with embodiments;

FIG. 31 shows radiofrequency (RF) predicted versus measured values for glucose concentration in a live bioreactor cell culture, in accordance with embodiments;

FIG. 32A-32D shows radiofrequency (RF) measured versus predicted values for glucose, lactate, viable cell density (VCD), and ammonia in a live bioreactor culture, in accordance with embodiments;

FIG. 33A shows a top perspective view of a radiofrequency sensor system, in accordance with embodiments;

FIG. 33B shows a bottom perspective view of a radiofrequency sensor system, in accordance with embodiments;

FIG. 34A shows a schematic of a radiofrequency sensor probe and its holder, in accordance with embodiments;

FIG. 34B shows a perspective view of an isolated radiofrequency sensor probe, in accordance with embodiments;

FIG. 34C shows a cross-sectional view of the isolated radiofrequency sensor probe, in accordance with embodiments;

The figures may not be to scale in absolute or comparative terms and are intended to be exemplary. The relative placement of features and elements may have been modified for the purpose of illustrative clarity. Where practical, the same or similar reference numbers denote the same or similar or equivalent structures, features, aspects, or elements, in accordance with one or more embodiments.

### DETAILED DESCRIPTION

Disclosed herein are systems (e.g., radiofrequency sensor systems) and devices (e.g., radiofrequency sensor introducers) useful in measuring one or more analytes of interest in a fluid medium (e.g., a liquid cell culture or fermentation medium). Systems and devices disclosed herein can comprise one or more radiofrequency sensors. In some implementations, a system or device disclosed herein can be configured to measure an analyte in a fluid medium, for instance, using a radiofrequency signal. In some implementations, a measured aspect of an analyte of interest in a fluid (e.g., a bioreactor cell culture medium), such as a presence, an absence, or a concentration of the analyte of interest in a bioprocess container (e.g., relative to a predetermined levels or tolerances) can be used to determine a condition or status of a bioprocess (e.g., a bioreactor cell culture) or to determine and/or effectuate an intervention with respect to a bioprocess (e.g., a bioreactor or fermentor cell culture). Measurement and/or analysis of the amount (e.g., presence, absence, or concentration) of one or more analytes of interest in a fluid medium of a bioprocess container (e.g., bioreactor) using a radiofrequency signal passed through the fluid medium, as described herein, can avoid the need to extract and analyze portions of the cell culture medium to measure the analyte(s) of interest. In some implementations, this can reduce a risk of contamination of the bioreactor during sampling and/or increase feedback data frequency.

In some implementations, systems, methods, and devices are disclosed herein for radiofrequency measurement of one or more analytes in a fluid medium of a bioprocess container that can obviate adverse effects of removing fluid medium for analysis. As such, removal of even small amounts of fluid medium for analysis can be avoided in bioprocess containers having small fluid volume capacities or low fluid medium volumes. In some embodiments, systems, methods, and devices useful in radiofrequency measurement of one or more analytes in a fluid medium of a bioprocess container can obviate the high costs of purchasing and maintaining expensive analytical equipment, such as mass spectrometry systems, Raman spectroscopy systems, Fourier-transform infrared (FTIR) spectroscopy systems, or enzyme-based chemical biosensors.

Systems, devices, and methods for measuring one or more analytes in a fluid medium of a bioprocess container using a radiofrequency signal, as described herein, can allow for real-time measurement of the one or more analytes in the fluid medium of a bioprocess container (e.g., a cell culture medium or in-process fluid) without interrupting the operation or conditions within the bioprocess container. As disclosed herein, multiple radiofrequency sensors can be utilized to measure one or more analytes in a plurality of bioprocess containers and/or in a plurality of regions or chambers of a single bioprocess container, including in parallel. In some embodiments, systems, devices, and methods can comprise operation of bioreactor equipment (e.g., pumps, valves, mixers, etc.) to maintain or correct (e.g., based at least in part on one or more parameter thresholds and/or parameter tolerances) one or more conditions in the bioreactor based at least in part on the radiofrequency measurement of the one or more analytes of interest. In some embodiments, such operation of bioprocess equipment can be automated, e.g., based on one or more radiofrequency signals passed through the fluid medium.

In some implementations, a radiofrequency sensor of a radiofrequency sensor system can be configured to transmit a radiofrequency signal (e.g., a radiofrequency transmission signal) through a fluid medium to measure an analyte in the fluid medium. In some implementations, transmitting a radiofrequency signal through a fluid medium comprising an analyte can be useful in measuring the analyte. This can be achieved by determining a change in an amplitude of the radiofrequency signal and/or a phase shift of the radiofrequency signal between the transmitted radiofrequency signal and the radiofrequency signal received after the signal has passed through the fluid medium (e.g., wherein the change(s) in the radiofrequency signal are caused by the radiofrequency signal interacting with the analyte as it passes through the fluid medium).

In some implementations, a radiofrequency signal transmitted through a fluid medium by a radiofrequency sensor can comprise a single radiofrequency signal frequency, a plurality of discrete radiofrequency signal frequencies, or one or more continuous ranges of radiofrequency signal frequencies. In some implementations, interaction of a radiofrequency signal with one or more analytes of interest (within a fluid medium) can change the transmitted radiofrequency signal. Such changes in the signal can include one or more changes in radiofrequency signal amplitude and/or phase at one or more radiofrequency signal frequencies (or ranges of frequencies). This can be detected at a radiofrequency receiver of radiofrequency sensor and transformed into a digital or analog resultant signal. The resultant signal can comprise a pattern characteristic of, or specific to, the presence or absence of an analyte in a fluid medium, the presence or absence of a specific combination of analytes in the fluid medium, a concentration of a specific analyte in the fluid medium, or concentration(s) of one or more specific analytes of a combination of analytes in the fluid medium. In some embodiments, a pattern can comprise one or more amplitude changes and/or one or more phase shifts relative to a reference signal, (e.g., initial calibration). In some embodiments, a pattern can comprise a magnitude of one or more amplitude changes relative to a reference signal. The magnitude of the one or more amplitude changes can be an absolute magnitude or a relative magnitude, for example, with respect to an input signal magnitude. In some embodiments, a pattern can comprise a magnitude of one or more phase shifts relative to a reference signal. The magnitude of the one or more phase shifts can be an absolute magnitude or a relative magnitude. In some embodiments, the magnitude of the phase shift is relative to a reference signal. Detection of a pattern of one or more amplitude changes and or phase shifts in a resultant signal, for example relative to a reference signal, can be useful in identifying the presence and/or concentration of one or more analytes of interest in a fluid medium. In various embodiments, systems and/or devices disclosed herein can be useful in measuring an analyte (e.g., determining a concentration of an analyte) in a fluid medium based on a change in amplitude and/or phase shift of a radiofrequency signal passed through the fluid medium. For example, a radiofrequency sensor 102 of the system or device can be used. In some implementations, the use of a radiofrequency signal, as described herein, can avoid a need for directly sampling (e.g., removing a portion of) the fluid medium to measure an analyte in a fluid medium of a bioprocess system. In some implementations, systems and devices can be useful in reducing the technical complexity and/or cost of measuring an analyte in a bioprocess system. In some implementations, systems and devices described herein can be useful in enabling feedback control of elements and aspects of the bioprocess.

A radiofrequency (RF) sensor system or a radiofrequency sensor introducer 120 can comprise one or more radiofrequency sensors. A radiofrequency sensor (“RF sensor”) can comprise a radiofrequency transmitter (“RF transmitter”) and a radiofrequency receiver (“RF receiver”). In embodiments, an RF transmitter of an RF sensor can be used to transmit a radiofrequency transmission signal through fluid (e.g., a fluid medium, such as a cell culture medium comprising an analyte of interest). An RF receiver of an RF sensor can be used to receive a radiofrequency signal transmitted through a fluid medium from a radiofrequency transmitter of the radiofrequency sensor. In some cases, a radiofrequency transmission signal can change as a result of interacting with a component of a fluid medium through which it is transmitted, for instance, wherein the component is one or more analytes of interest in a fluid medium of a bioprocess container. In some implementations, a radiofrequency signal received by the RF receiver of the RF sensor can be transmitted (e.g., as a resultant signal) by the radiofrequency receiver to a computer system controller comprising a processor for analysis.

As described herein, a radiofrequency transmission signal transmitted through a fluid medium can be altered in one or more aspect before reaching an RF receiver. For example, this can be due to interaction of the transmitted radiofrequency signal with the fluid medium and/or the one or more analytes of interest in the fluid medium). The altered radiofrequency transmission signal can be received as a resultant signal. In some cases, a radiofrequency transmission signal transmitted through a fluid medium can be unaltered in one or more aspect when it reaches a radiofrequency receiver. For example, this can be due to a lack of interaction of the transmitted radiofrequency signal with the fluid medium and/or the one or more analytes of interest in the fluid medium (e.g., due to a lack of the one or more analytes). The unaltered radiofrequency transmission signal can be received as a resultant signal. In some cases, one or more aspects of the resultant signal received at and/or transmitted by a radiofrequency receiver can be different than one or more corresponding aspects of a radiofrequency transmission signal transmitted by a radiofrequency transmitter of an RF sensor or RF bioreactor sensor system. Such differences can be analyzed (e.g., by a processor) for determining a concentration of one or more analytes in the fluid medium.

An RF sensor system 100 disclosed herein can comprise a radiofrequency sensor 102 (RF sensor) useful in measuring an analyte of interest in a fluid 141 of a bioprocess container 140 (e.g., a bioreactor, cell culture flask, or vial). A fluid 141 of a bioprocess container 140 can comprise a cell culture fluid, such as a cell culture medium, and/or an in-process fluid, such as a buffer, a drug product, and/or a drug substance containing fluid. By way of example and not by limitation, fluid 141 can include one or more biocomponents, including fluids, solids, mixtures, solutions, and suspensions including, but not limited to, bacteria, fungi, algae, plant cells, animal cells, white blood cells, T-cells, cell media, protozoans, nematodes, plasmids, viral vectors, blood, plasma, organelles, proteins, nucleic acids, lipids, plasmids, carbohydrates, and/or other biological components, and the like. Examples of some common biological components that are grown in fluid 141 include E. coli, yeast, bacillus, and CHO cells. Fluid 141 can also comprise cell-therapy cultures and cells and microorganisms that are aerobic or anaerobic and adherent or non-adherent. Different media compositions known in the art can be used to accommodate the specific cells or microorganisms grown and the desired end product. In some implementations, bioprocess container 140 is configured for biological reactions, including but not limited to, growing cells or other biological components. In example embodiments, bioprocess container 140 can also comprise or be substituted with one or more bioreactors, fermenters, mixers, storage vessels, fluid management systems, cell culture equipment, centrifuges, centrifugal separators, chromatography units, mixers, homogenizers, magnetic processing units, blood separating devices, biocomponent filtering devices, biocomponent agitators or any other device designed for growing, mixing or processing cells and/or other biological components. It is also appreciated that bioprocess container 140 can comprise any conventional type of bioreactor, fermenter, or cell culture devices such as a stirred-tank reactor, rocker-type reactor, paddle mixer reactor, or the like.

In various embodiments, an RF sensor 102 can comprise a radiofrequency transmitter 104 (RF transmitter) and/or a radiofrequency receiver 106 (RF receiver). In some embodiments, an RF receiver 106 of a radiofrequency sensor system or device can be a separate component (e.g., a first radiofrequency antenna or a first RF sensor) from an RF transmitter 104 of the system or device (e.g., a second radiofrequency antenna or a second RF sensor). In some embodiments, a single radiofrequency component (e.g., radiofrequency antenna or RF sensor) can function as and/or can be operated as both an RF transmitter 104 described herein and an RF receiver 106 described herein. For example, an RF sensor can be configured and/or used to receive a radiofrequency signal after transmitting the signal into and/or through at least a portion of a fluid medium of a bioprocess container described herein. The RF transmitter 104 can be separated from the RF receiver 106 by a sensor separation distance 116. In some embodiments, the RF transmitter of the RF sensor can be configured to transmit a radiofrequency signal (e.g., a radiofrequency transmission signal) to the RF receiver 106 (e.g., across the sensor separation distance 116). The RF receiver 106 can be configured to receive the radiofrequency signal transmitted by the RF transmitter 104. In some cases, a portion of fluid 141 (e.g., a culture medium comprising an analyte of interest) can be disposed in a space between RF transmitter 104 and the RF receiver 106 of the RF sensor 102. In some embodiments, an RF sensor 102 can be configured to transmit the radiofrequency signal through the portion of the fluid disposed between the RF transmitter 104 and the RF receiver 106.

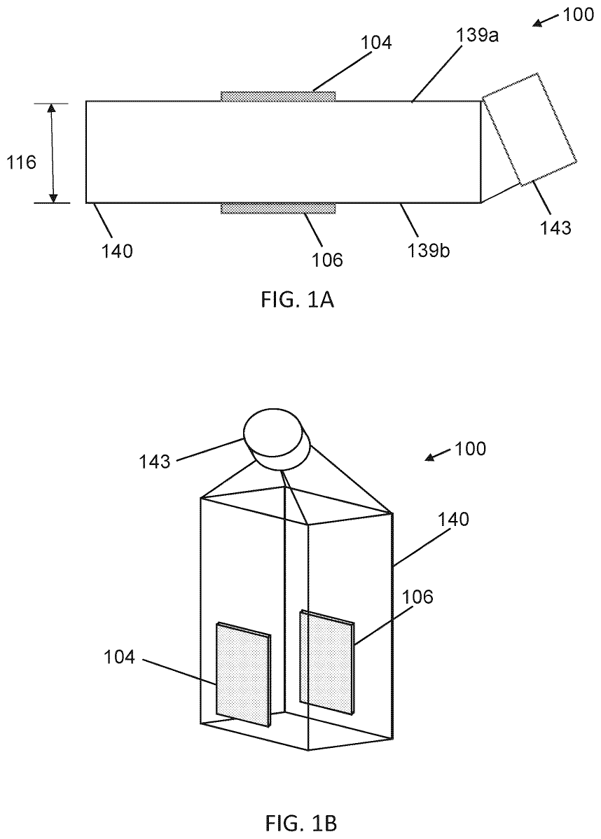

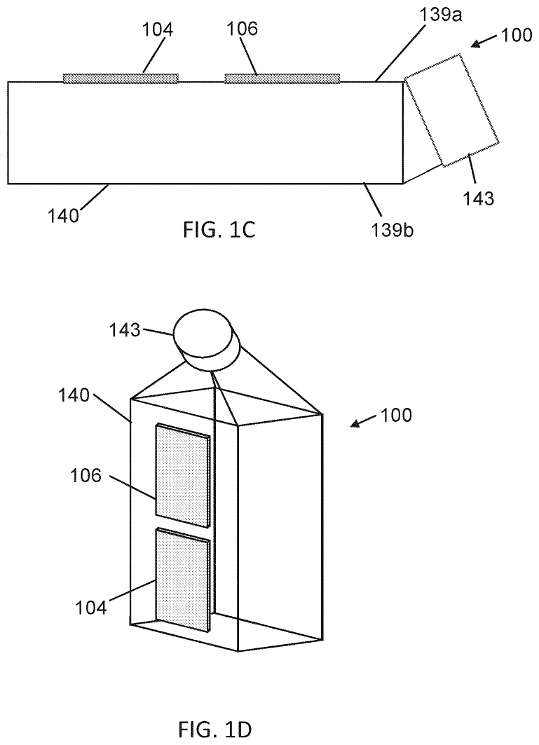

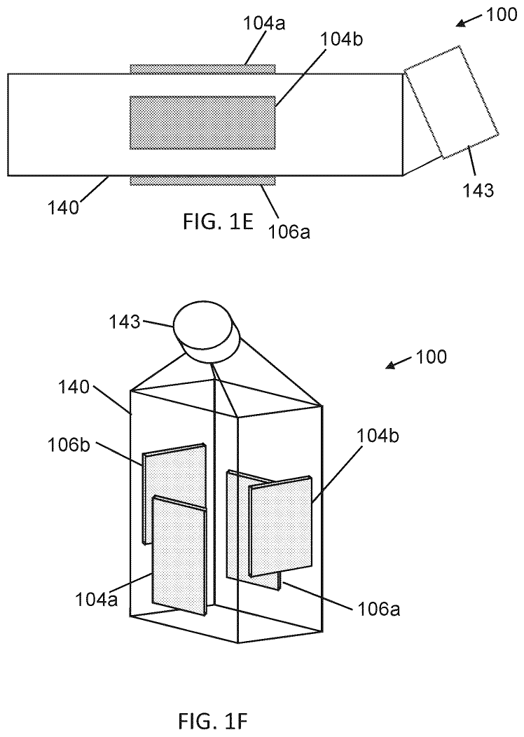

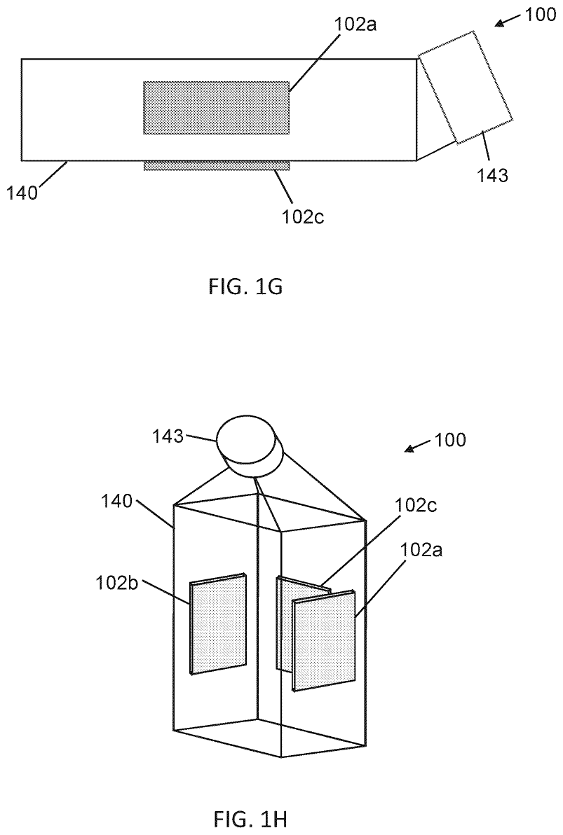

Turning to FIGS. 1A-1H, the one or more RF sensors 102 of an RF sensor system, which may comprise an RF transmitter 104, an RF receiver 106, or a radiofrequency antenna (RF antenna) configured to serve as both an RF transmitter and an RF receiver, can be disposed in contact with, adjacent to, or in close proximity to a bioprocess container 140 or a portion thereof (e.g., an outer surface of a wall 139 of the bioprocess container). In some embodiments, the one or more RF sensors 102 can be integrated into the bioprocess container 140 or a portion thereof (e.g., a wall 139 of the bioprocess container). In some embodiments, the one or more RF sensors 102 (e.g., RF transmitter 104 and/or RF receiver 106) can be adhered to a portion of the bioprocess container 140 (e.g., an outer surface of a wall 139 of the bioprocess container). For example, an RF transmitter 104 and/or an RF receiver 106 can be adhered to a surface (e.g., an outer surface) of a bioprocess container 140 using a glue or an epoxy. In some cases, the one or more sensors 102 can be permanently adhered to the bioprocess container 140. In some embodiments, the one or more sensors 102 can be reversibly adhered to the bioprocess container 140, for instance, to remove the one or more sensors 102 from a single-use bioprocess container after use. In some embodiments, a bioprocess container 140 can comprise markings on one or more surfaces of the bioprocess container and/or one or more recessed regions or raised regions of the bioprocess container to indicate the location(s) where an RF sensor should be placed for efficient and/or reproducible RF sensor system use. In some embodiments, the one or more RF sensors 102 (e.g., RF transmitter 104 and/or RF receiver 106) can be coupled to the bioprocess container 140 or portion thereof. For example, an RF transmitter 104 and/or an RF receiver 106 can be coupled to an outer surface of the bioprocess container using a sleeve or a fastener, such as a clip or a screw. In some embodiments, the one or more RF sensors 102 can be uncoupled from the bioprocess after use, for example, so that the one or more RF sensors 102 may be used again. Reuse of the one or more RF sensors 102 can greatly improve the cost efficiency of the RF sensor system.

FIG. 1A and FIG. 1B show RF sensor systems 100 comprising an RF transmitter 104 disposed on an outer surface of a first wall 139a of a bioprocess container (e.g., a cell culture flask) and an RF receiver 106 disposed on an outer surface of a second, opposing wall 139b of the bioprocess container. In some embodiments, such a configuration has the advantage of standardizing the distance 116 between the sensors, for example, when the bioprocess container 140 is a cell culture vessel with a standard height or width. In some embodiments, the bioprocess container 140 can have a cap 143, for instance, to cover an orifice through which a fluid can be introduced and/or removed from the bioprocess container.

In addition to or instead of receiving, detecting, or measuring with an RF receiver 106 an RF signal that has been transmitted directly through a fluid of a bioprocess container by an RF transmitter 104, an RF sensor 102 (e.g., an RF antenna functioning as an RF receiver 106) can be used to receive, detect, or measure an RF signal that has reflected back (e.g., directly or at an oblique angle) from a fluid after interacting with at least a portion of the fluid. FIG. 1C and FIG. 1D show embodiments wherein an RF transmitter 104 and an RF receiver 106 are disposed near or in contact with the same wall of the bioprocess container 140. The RF receiver 106 can be configured to receive, detect, or measure all or a portion of an RF signal transmitted through at least a portion of a fluid contained within the bioprocess container 140 after the RF signal has been transmitted into the fluid by an RF transmitter 104 disposed on the same wall of the bioprocess container. In some cases, positioning the RF transmitter 104 on the same wall of the bioprocess container (or same wall of a housing 137 as described herein) can aid in isolating the RF sensors from radiofrequency interference. In some embodiments, the RF receiver 106 can be disposed at an angle (e.g., a perpendicular angle or oblique angle) to the RF transmitter 104 to receive a reflected component of an RF signal transmitted by the RF transmitter 104 into a fluid contained within the bioprocess container 140. For example, RF receiver 106 can be adhered to a first wall of a bioprocess container 140 that is perpendicular to a second wall of the bioprocess container 140 to which RF transmitter 104 is adhered and can be configured to receive a portion of an RF signal transmitted into a fluid of the bioprocess container that is reflected to the RF receiver 106.

FIG. 1E and FIG. 1F show an arrangement of four RF sensors 102 of an RF sensor system in which a first RF transmitter 104a is disposed on a first wall of the bioprocess container 140, a first RF receiver 106a is disposed on a second wall of the bioprocess container 140 opposite the first wall, a second RF transmitter 104b is disposed on a third wall of the bioprocess container 140, and a second RF receiver 106b is disposed on a fourth wall of the bioprocess container 140 that is opposite the third wall. In some embodiments, the first pair of RF sensors 102 comprising the first RF transmitter 104a and the first RF receiver 106a can be used to assay a fluid 141 within the bioprocess container 140 in a first direction, and a second pair of RF sensors 102 comprising the second RF transmitter 104b and the second RF receiver 106b can be used to assay the fluid 141 in the second direction. In some cases, the separation distance between the first RF transmitter 104a and the first RF receiver 106a is different than the separation distance between the second RF transmitter 104b and the second RF receiver 106b.

FIG. 1G and FIG. 1H show an arrangement of three RF sensors (102a, 102b, and 102c) relative to a bioprocess container. One or more of RF sensors 102a, 102b, and 102c can be configured to receive a radiofrequency signal (RF signal) reflected back from a fluid within the bioprocess container 140. In some embodiments, an RF sensor 102 can be configured to transmit an RF signal into a portion of a fluid within bioprocess container 140 and then to receive at least a portion of the RF signal after it has been reflected back to the RF sensor 102 from the fluid. In some embodiments, an RF sensor 102 can be configured to receive (e.g., detect or measure) at least a portion of an RF signal transmitted into a fluid of the bioprocess container 140 by a different RF sensor 102 of the RF sensor system 100. For example, RF sensor 102c, which may be oriented at an angle relative to RF sensor 102a, can be configured to receive (e.g., detect or measure) all or a portion of an RF signal transmitted into a fluid of bioprocess container 140 by RF sensor 102a (e.g., after the portion of the RF signal has been reflected or scattered toward RF sensor 102c by interaction with the fluid). Additionally, RF sensor 102b, which can be disposed directly opposite or substantially directly opposite RF sensor 102a (e.g., on an outer surface of an opposing wall of bioprocess container 140), may be configured to receive a portion of the same RF signal (or a different RF signal) transmitted by RF sensor 102a through the fluid of the bioprocess container.

As described herein, one or more RF sensors 102 (e.g., one or more RF transmitters 104 and/or one or more RF receivers 106) can be in communication with a computer system or component thereof (e.g., a vector network analyzer (VNA)) of an RF sensor system 100. In some embodiments, the one or more RF sensors 102 (e.g., the one or more RF transmitters 104 and/or the one or more RF receivers 106) can be in wireless communication (e.g., via a wireless communication element 110 of the RF sensor 102) with the computer system or component thereof. In some cases, data can be transferred to and/or from the one or more RF sensors 102 via the wireless communication connection. In some embodiments, the one or more RF sensors 102 (e.g., the one or more RF transmitters 104 and/or the one or more RF receivers 106) can be in communication with the computer system or component thereof (e.g., a VNA of the computer system) via a cable or other wired communication connection. In some cases, data can be transferred to and/or from the one or more RF sensors 102 via the wired communication connection. For instance, data can be transmitted from the VNA to RF transmitter 104 via a wired connection. In some embodiments, data can be transmitted from the RF receiver to the VNA via a wired connection. In some embodiments, a VNA of computer system 150 can be in wired or wireless communication with a controller of computer system 150 and in wired communication with one or both of RF transmitter 104 and RF receiver 106. It is contemplated that any RF sensor 102, any RF transmitter 104, and/or any RF receiver 106 disclosed herein can include a communication element 110 (or can be configured to or adapted to include a communication element 110), for example, to place the RF sensor(s) 102, RF transmitter(s) 104, and/or RF receiver(s) 106 in wired or wireless communication with the computer system or component thereof, whether or not the RF sensor, RF transmitter, or RF receiver is explicitly disclosed as having such configurations or capabilities in a given embodiment of this disclosure.

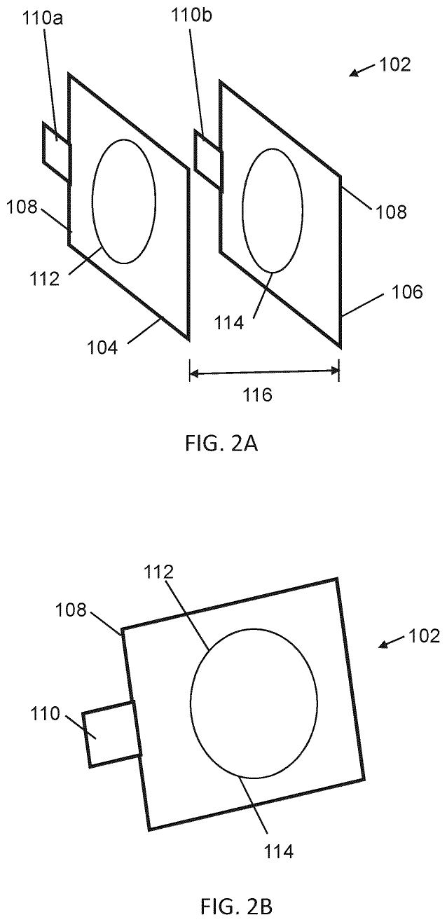

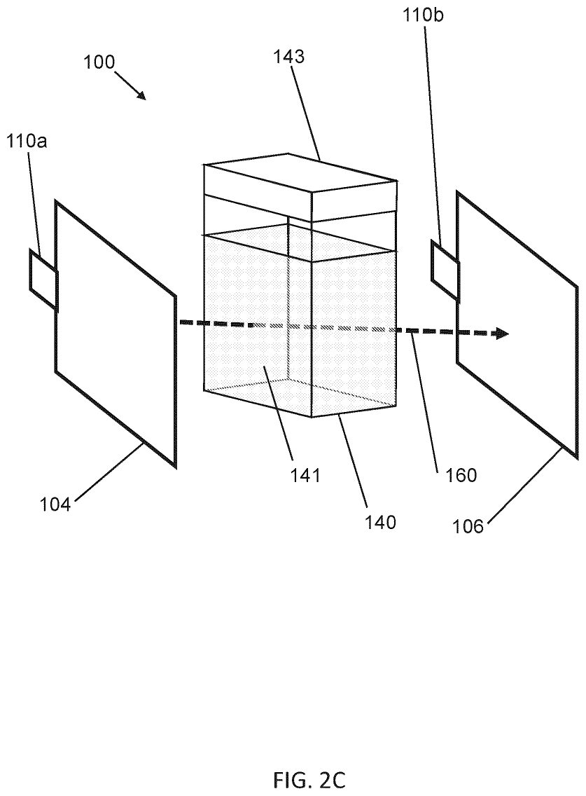

FIGS. 2A and 2B illustrate an RF transmitter 104 of an RF sensor 102 including a radiofrequency transmitter antenna 112 coupled to a substrate 108 (e.g., a semiconductor wafer). An RF sensor 102 can comprise one or more communication elements 110 for communicating via wired or wireless connection with a computer system controller 150. In some implementations, communications element 110 (e.g., 110a and/or 110b) can be a wired connection to one or more additional components of a system described herein. For example, communications element 110 (e.g., 110a and/or 110b) can be a component (e.g., a digital communications wire, such as a coaxial cable or USB cable) forming a wired connection between an RF component (e.g., RF transmitter 104 or RF sensor 102) and a computer system component (e.g., vector network analyzer (VNA)). In some embodiments, communications element 110 (e.g., 110a and/or 110b) can comprise a wireless transmitter and/or receiver. In some implementations, a communications element 110 comprising a wireless transmitter and/or receiver can be configured to transmit and/or receive data between a computer system described herein or a component thereof (e.g., a VNA) and an RF transmitter 104 or an RF sensor 102. Accordingly, data comprising a driver signal 164 and/or all or a portion of a resultant signal (e.g., received at an RF sensor 102) can be communicated to a computer system (e.g., in a process involving the data being received an analyzed by a VNA component of the computer system). In some embodiments, the radiofrequency transmitter antenna 112 can be electronically coupled to a communication element 110a, which may be coupled to the substrate 108. The RF receiver 104 of RF sensor 102 can include a radiofrequency receiver antenna 114 coupled to another substrate 108. In some embodiments, the radiofrequency transmitter antenna 112 can be electronically coupled to a communication element 110a, which may be coupled to the substrate 108. An RF transmitter 104 of RF sensor 102 can be arranged at the sensor separation distance 116 from RF receiver 106 of RF sensor 102. Fixing the RF transmitter 104 with respect to position and orientation relative to the position and orientation of RF receiver 106 can improve reproducibility and accuracy of radiofrequency measurements of analytes of interest. For example, such positioning and orientation can maintain a consistent volume through which a radiofrequency signal is transmitted before reaching the RF receiver 106 from the RF transmitter 104. In some embodiments, a rigid structure or structure (e.g., an introducer 120 or a wall 155 or other portion of housing 137 described herein) can be used to fix the position and/or orientation of the RF transmitter 104 and RF receiver 106. In some embodiments, the sensor separation distance 116 is less than 10 millimeters (mm), from 10 mm to 15 mm, from 15 mm to 20 mm, from 20 mm to 25 mm, from 25 mm to 30 mm, from 30 mm to 40 mm, from 40 mm to 50 mm, from 50 mm to 100 mm, from 100 mm to 200 mm, from 200 mm to 300 mm, or larger than 300 mm.

In some cases, a radiofrequency transmitter antenna 112 (which may be circular in shape) can be disposed entirely within a plane that is parallel to a plane entirely containing radiofrequency receiver antenna 114 (which may be circular in shape). In some cases, an RF sensor 102 can comprise an antenna that can be used as a transmitter antenna 112 and as a receiver antenna 114, for example, as shown in FIG. 2B. In some cases, a direction of transmission of a radiofrequency transmission signal from the RF transmitter 104 to the RF receiver 106 can be perpendicular to both of the respective planes entirely containing the RF transmitter 104 or the RF receiver 106. In some cases, a relative angle of orientation of an RF transmitter 104 or plane entirely encompassing the RF transmitter 104 to an RF receiver 106 or plane entirely encompassing the RF receiver 106 can be from 0 to 5 degrees, from 5 to 10 degrees, from 10 to 15 degrees, from 15 to 20 degrees, from 20 to 25 degrees, from 25 to 30 degrees, from 30 to 45 degrees, from 45 to 60 degrees, or from 60 to 90 degrees. In some cases, an RF transmitter 104 can be disposed on an adjacent wall or on the same wall of the bioprocess container 140.

In some embodiments, one or more RF sensors 102 of RF sensor system 100 do not need to be in direct contact with the bioprocess container 140. For example, one or more RF sensors 102 can be positioned relative to the bioprocess container so as to be able to transmit an RF signal to and/or receive an RF signal from (e.g., detect or measure) a fluid within a bioprocess container 140 without the one or more RF sensors being in contact with the bioprocess container 140. FIG. 2C shows an RF sensor system 100 wherein an RF transmitter 104 and an RF receiver 106 of the system are configured to transmit an RF signal 160 through a fluid 141 within a bioprocess container 140 having a cap 143, for instance, to analyze a portion of the fluid 141 (e.g., one or more analytes of interest in the fluid 141) as described herein. In some cases, RF transmitter 104 can comprise a first communication element 110a (e.g., for receiving a driver signal from a computer system to produce a desired RF signal). In some cases, RF receiver 106 can comprise a second communication element 110b (e.g., for transmitting a detected or measured resultant RF signal produced from interaction of the transmitted RF signal with the fluid or analyte of interest thereof).

As described herein at least a portion of a fluid medium 141 of a bioprocess container can be disposed between the RF transmitter antenna 112 and the RF receiver antenna 114, for example, so that a radiofrequency transmission signal transmitted from the RF transmitter antenna 112 to the RF receiver antenna 114 passes through the portion of the fluid medium. In some cases, an RF transmitter antenna 104 can be circular in shape. In some cases, an RF transmitter antenna 112 can have a diameter of 0.1 to 1 mm, 1 to 2 mm, 2 mm to 5 mm, 5 mm to 10 mm, or larger than 10 mm. In some cases, an RF receiver antenna 114 can be circular in shape. In some cases, an RF receiver antenna 114 can have a diameter of 0.1 to 1 mm, 1 to 2 mm, 2 mm to 5 mm, 5 mm to 10 mm, or larger than 10 mm. In some implementations, the RF receiver antenna 106 can have an identical configuration as the RF transmitter antenna 104. In some implementations, the RF receiver antenna 106 can have a different configuration than the RF transmitter antenna 104. A radiofrequency signal passing through the fluid medium can interact with one or more analytes in the fluid medium, and the interaction of the radiofrequency signal with the one or more analytes can change or alter one or more aspects (e.g., amplitude or phase modulation) of all or a portion of the radiofrequency signal, for example, as described herein. In some cases, an RF sensor 102 can be configured to transmit a radiofrequency through one or more walls of the bioprocess container 140.

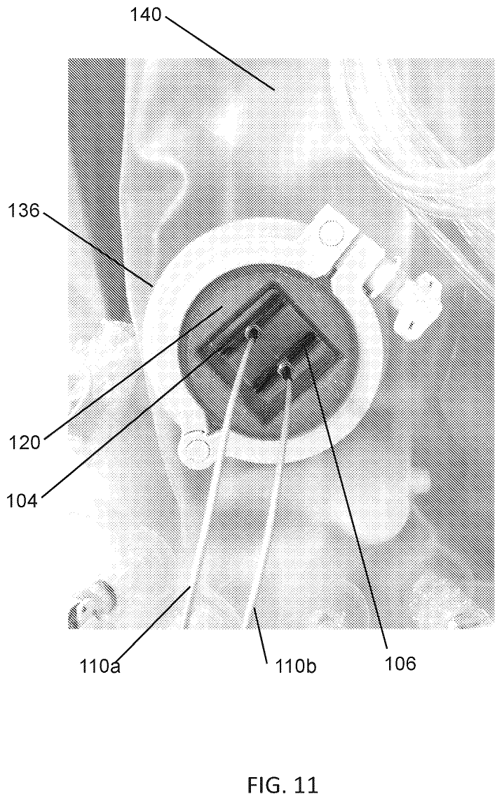

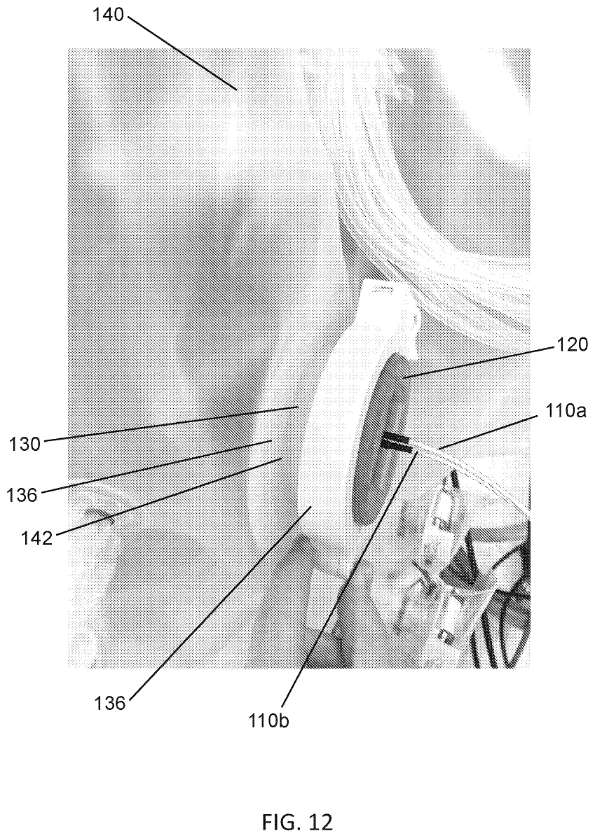

FIG. 2C illustrates an embodiment of the radiofrequency sensor system 100 including the RF sensor 102 passing a radiofrequency signal through at least one wall of the bioprocess container 140. In some embodiments, the RF sensor 102 can be configured to transmit a radiofrequency signal through an entire width, length, or thickness of the bioprocess container 140. For example, as shown in FIG. 2C, a radiofrequency signal can be transmitted along a radiofrequency signal path 160 from an RF transmitter 104, through a first wall of the bioprocess container 140, through at least a portion of the fluid medium 141 disposed within the bioprocess container 140, through a second wall of the bioprocess container 140, and on to the RF receiver 106. In some cases, an RF transmitter 104 and an RF receiver 106 of an RF sensor 102 can be disposed on opposite sides of a portion of the bioprocess container 140. For example, RF transmitter 104 and an RF receiver 106 of an RF sensor 102 can be disposed on opposite sides of a tube, channel, or chamber of the bioprocess container 140. In some embodiments, RF transmitter antenna 104 and RF receiver antenna 106 shown in FIG. 11 can be implemented using an introducer 120.

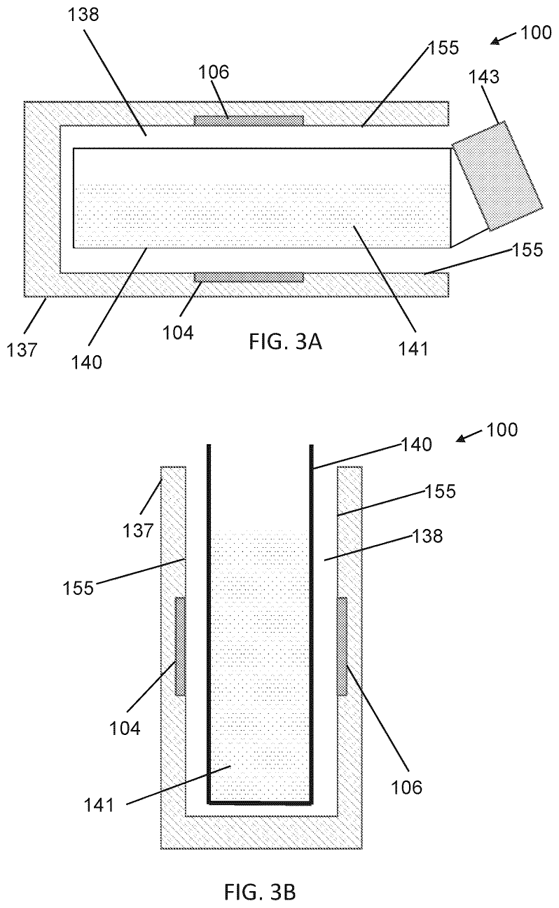

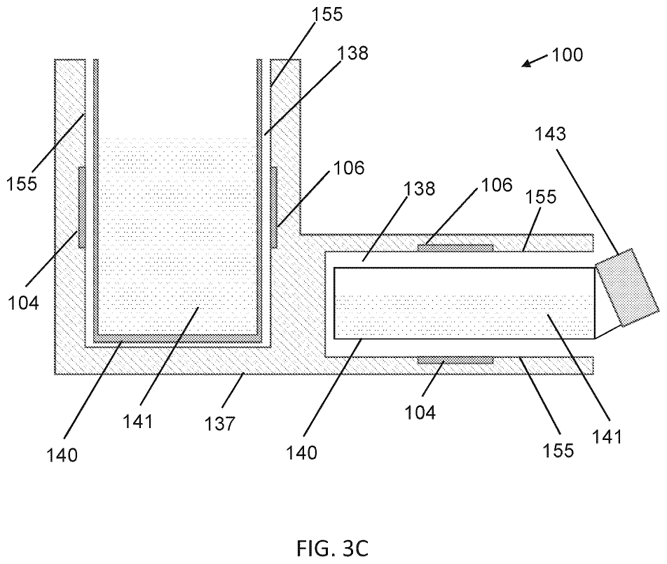

Turning to FIGS. 3A-3C, one or more RF sensors 102 (e.g., one or more RF transmitters 104 and/or one or more RF receivers 106) can be adhered to, coupled to, or an integrated part of a housing 137 of RF sensor system 100. A housing 137 of RF sensor system 100 can comprise a cavity 138 shaped and sized to receive or house all or a portion of a bioprocess container 140. In some cases, housing 137 can comprise two or more opposing walls 155 to at least partially define one or more cavities 138 of housing 137. In some embodiments, the two or more walls 155 can be configured to receive a bioprocess container in a cavity 138 at least partially defined by the two or more walls 155. In some embodiments, a bioprocess container 140 can be slideably inserted horizontally into a cavity 138 of housing 137, for instance, as shown in FIG. 3A. In some embodiments, a bioprocess container 140 can be slideably inserted vertically into a cavity 138 of housing 137, for instance, as shown in FIG. 3B. Optionally, housing 137 can comprise means for securing at least a portion of bioprocess container 140 in housing 137, such as a covering or door to fully or partially close an opening to cavity 138 and/or a clamp or a spring within the cavity 138 and biased against the bioprocess container, or rubber pad inside of the cavity to increase friction of between the bioprocess container 140 and the housing 137. All or a portion of housing 137 (e.g., a wall 155 of housing 137) can comprise a material or structure for electromagnetically isolating all or a portion of bioprocess container 140 or cavity 138 from outside electromagnetic signals (e.g., other than those introduced by an RF sensor 102 or RF transmitter 104 into the bioprocess container 140 or cavity 138). For instance, all or a portion of housing 137 can comprise a metal material or metal mesh for attenuating or blocking electromagnetic signals not originating from within the cavity. FIG. 3C shows an embodiment of an RF sensor system 100, wherein a housing 137 of the system comprises a plurality of cavities 137 configured to receive bioprocess containers 140.

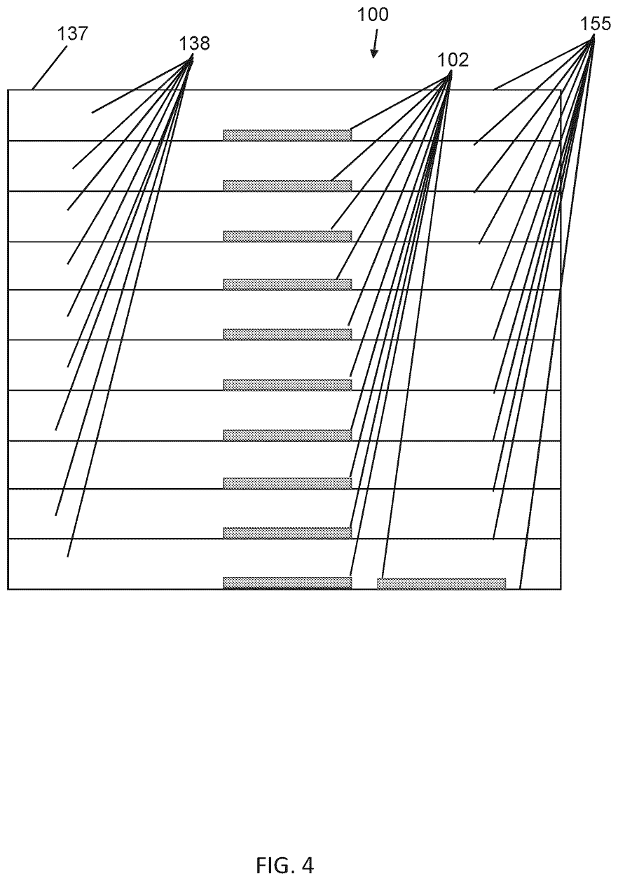

FIG. 4 shows an embodiment of an RF sensor system 100, wherein a housing 137 comprises a plurality of cavities 138 defined by a plurality of walls 155 and configured to receive one or more bioprocess containers each, wherein the housing further comprises a plurality of RF sensors 102 configured to transmit an RF signal into and/or receive an RF signal from a fluid of a bioprocess container 140 positioned in a cavity 138 of housing 137.

The one or more RF sensors 102 (e.g., one or more RF transmitters 104 and/or one or more RF receivers 106) can be adhered to, coupled to, or an integrated into one or more walls 155 of housing 137. For instance, a housing 137 can comprise an RF transmitter 104 adhered to, coupled to, or integrated on a first wall 155 of housing 137 and an RF receiver 106 adhered to, coupled to, or integrated on a second, opposing wall 155 of housing 137. An RF transmitter 104 adhered to, coupled to, or integrated on a first wall of housing 137 and an RF receiver 106 adhered to, coupled to, or integrated on a second wall of housing 137 can be configured to transmit an RF signal through a fluid 141 of a bioprocess container 140 (e.g., by positioning and/or orienting the RF transmitter 104, RF receiver 106, and/or wall(s) 155 of housing 137). In some embodiments, an RF transmitter 106 adhered to, coupled to, or integrated on a first wall of housing 137 and an RF receiver 106 adhered to, coupled to, or integrated on a second wall of housing 137 can be configured to receive an RF signal reflected from a fluid 141 of a bioprocess container 140 (e.g., by positioning and/or orienting the RF transmitter 104, RF receiver 106, and/or wall(s) 155 of housing 137). In some embodiments, an RF receiver 106 can be positioned in or on (e.g., adhered to, coupled to, or integrated in) the same wall 155 of housing 137, for instance to receive a reflected RF signal transmitted into a fluid 141 by the RF transmitter 104. By adhering, coupling, or integrating an RF transmitter 104 and RF receiver 106 onto one or more portions of housing 137, separation distance between the RF sensors can be maintained, even as bioprocess containers 140 are inserted into, removed into, or exchanged from a cavity 138. Accordingly, less time is required to apply and/or replace the RF sensors to achieve efficient measurement of analytes of interest in fluids of bioprocess containers placed into the cavity 138. In some cases, such configurations can also reduce the likelihood of damage or misalignment from physical contact between the bioprocess container 140 and one or more RF sensors 102 (e.g., if the one or more RF sensors 102 are positioned flush with a wall 155 of housing 137 or internal to a wall 155 of housing 137. In some embodiments, communication elements 110 can be routed through walls 155 of housing 137 and, optionally, collected at a local transmitter in communication with the computer system. Alternatively, the housing 137 may comprise all or a portion of the computer system (e.g., a processor, computer memory, and a VNA module, or only a VNA module) locally so as to allow the housing 137 to process RF signals locally. In some cases, housing 137 can comprise a display and/or user input interface.

In some cases, the radiofrequency signal transmitted through the portion of the fluid medium 141 disposed between the RF transmitter 104 and the RF receiver 106 can be changed or altered as it passes through the fluid medium 141. In some cases, the radiofrequency signal can be changed or altered in a different manner or to a different degree based on the composition of the fluid medium 141. For example, a radiofrequency signal may be changed or altered in a different respect (e.g., with respect to amplitude or with respect to phase modulation) or to a different degree if the fluid medium contains a first set of solutes (e.g., comprising one or more analytes of interest) as compared to a the manner or degree to which a radiofrequency signal would be changed or altered if the same fluid medium contained a second set of solutes. For example, one or more analytes of interest may not be present in the second set of solutes and/or one or more additional analytes of interest not present in the first set of solutes may be present in the second set of solutes. In some cases, a radiofrequency signal can be changed or altered in a different manner or to a different degree if the radiofrequency signal is transmitted through a fluid medium containing a first concentration of one or more analytes of interest than if the radiofrequency signal is transmitted through a fluid medium 141 comprising a second concentration of the same one or more analytes of interest. Accordingly, different configurations of radiofrequency signals (e.g., modulation of amplitude and/or frequency) can be used to achieve optimal detection of different combinations of analytes of interest in the fluid medium 141. In some implementations, interaction of the radiofrequency signal with the one or more analytes of interest can result in a decreased signal amplitude at one or more frequencies or frequency ranges. In some implementations, interaction of the radiofrequency signal with the one or more analytes of interest can result in a delayed phase modulation (e.g., positive phase shift) at one or more frequencies or frequency ranges. In some implementations, interaction of the radiofrequency signal with the one or more analytes of interest can result in a forward phase modulation (e.g., negative phase shift) at one or more frequencies or frequency ranges. In some cases, a system 100 disclosed herein can be useful in measuring (e.g., detecting) a presence, absence, or concentration of one or more analytes of interest in a fluid medium of a bioprocess container 140, such as a bioreactor chamber. For example, a system 100 disclosed herein can be used to measure a presence, absence, or concentration of one or more analytes in a fluid medium 141 of a bioprocess container. In some cases, the fluid medium 141 can comprise a cell culture fluid. In some cases, the fluid medium 141 can comprise an in-process fluid, such as a buffer, a drug product, and/or a drug substance containing fluid. This can be done by comparing one or more characteristics of a first radiofrequency signal (e.g., an amplitude or phase shift of all or a portion of the signal) that has been transmitted through at least a portion of the fluid medium 141 to one or more corresponding characteristics of a second reference radiofrequency signal obtained by passing a radiofrequency signal through a fluid medium containing a known amount of the one or more analytes of interest. In some cases, an interpolated radiofrequency signal response can be determined between known values. For example, the first and second reference radiofrequency signals can have the same amplitude and/or frequency.

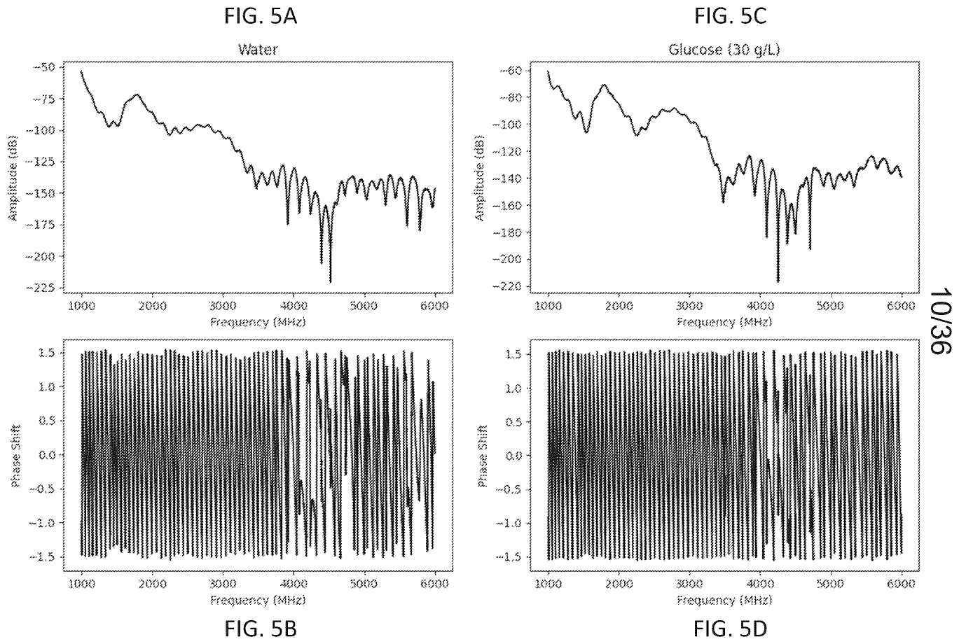

A radiofrequency signal can comprise a frequency or range of frequencies. In some cases, a radiofrequency signal (e.g., a radiofrequency transmission signal or a resultant signal) can comprise a range of frequencies of from 1 Hz to 10 GHz, from 1 Hz to 9 GHZ, from 1 Hz to 8 GHz, from 1 Hz to 7 GHZ, from 1 Hz to 6 GHz, from 1 Hz to 5 GHZ, 1 Hz to 4 GHZ, from 1 Hz to 3 GHZ, from 1 Hz to 2 GHz, from 1 Hz to 1 GHz, from 25 MHz to 10 GHz, from 25 MHz to 9 GHz, from 25 MHz to 8 GHz, from 25 MHz to 7 GHz, from 25 MHz to 6 GHz, from 25 MHz to 5 GHz, 25 MHz to 4 GHZ, from 25 MHz to 3 GHZ, from 25 MHz to 2 GHz, or from 25 MHz to 1 GHz. A radiofrequency signal of a system, device, or method disclosed herein (e.g., a radiofrequency transmission signal prior to transmission through a fluid medium, a radiofrequency transmission signal after transmission through a fluid medium, or a resultant signal produced by an RF receiver 106 after a radiofrequency transmission signal is received at the RF receiver 106) can be a periodic signal having a constant or variable frequency (e.g., wherein the wavelength of the radiofrequency signal is constant or variable). A radiofrequency can also comprise an amplitude and a phase modulation (e.g., a phase modulation relative to a reference radiofrequency signal, such as the phase modulation of the radiofrequency signal before transmission through a fluid medium comprising an analyte of interest). As described herein, a radiofrequency signal can be changed or altered with respect to amplitude and/or phase modulation of at least a portion of the radiofrequency signal when passed through a fluid medium, for example, as a result of interaction with the fluid medium and/or one or more analytes of interest in the fluid medium. For example, a radiofrequency passing through a fluid medium comprising an analyte of interest can be changed with respect to signal amplitude and/or phase modulation at a specific frequency of the radiofrequency signal or at one or more ranges of frequencies of the radiofrequency signal (e.g., wherein the radiofrequency signal comprises a variable frequency, for instance, as shown in FIGS. 5A-5D).

FIG. 5A shows measured amplitude of a radiofrequency signal measured by a system described herein over a range of 1 GHz to 6 GHz in pure water. FIG. 5C shows measured amplitude of a radiofrequency signal measured by the same system over the same frequency range in an aqueous solution having 30 grams/liter (g/L) glucose. Differences between FIG. 5A and FIG. 5C show that amplitude losses characteristic of the presence of an analyte can be detected at different ranges of the 1 GHz to 6 GHz frequency sweep. For example, differences in the amplitude of the radiofrequency in the glucose solution compared to the pure water solution can be observed in the range of 3 GHz to 5 GHz in this experiment. FIG. 5B shows measured phase shift (in radians) of a radiofrequency signal measured by a system described herein over a range of 1 GHz to 6 GHZ in pure water. FIG. 5D shows measured phase shift (in degrees) over the same frequency range in an aqueous solution having 30 grams/liter (g/L) glucose. Differences between FIG. 5A and FIG. 5C show that phase shifts characteristic of the presence of an analyte can be detected at different ranges of the 1 GHz to 6 GHz frequency sweep. For example, differences in the amplitude of the radiofrequency in the glucose solution compared to the pure water solution can be observed in the range of 3.5 GHz to 5 GHz in this experiment. In some cases, one or more changes or alterations in an amplitude and/or phase modulation of a radiofrequency signal can be caused by interaction of the radiofrequency signal with a fluid medium and/or one or more analytes of interest in the fluid medium. In some cases, a change or alteration in a radiofrequency signal as it passes through a fluid medium comprising an analyte of interest can be indicative of and, in some cases, specific to the presence of the one or more analytes of interest in the fluid medium. In some cases, a radiofrequency signal changed or altered in a manner specific to the presence of, or interaction with, one or more analytes in a fluid medium can comprise a pattern useful in measuring the one or more analytes in the fluid medium. In some cases, such a pattern can comprise all or a portion of a radiofrequency received at an RF receiver 106 of an RF sensor 102 and/or all or a portion of a resultant signal transmitted to a controller 150 of a system, device, or method disclosed herein by an RF receiver 106, e.g., wherein the resultant signal is produced by the RF receiver 106 based at least in part on a radiofrequency transmission signal received by the RF receiver after having passed through a fluid medium.

In some cases, a resultant signal can be useful in measuring (e.g., determining a presence, absence, or concentration) of one or more analytes of interest in the fluid medium. For instance, a first resultant signal having a greater amplitude loss (e.g., a lower measured radiofrequency signal amplitude) at one or more specific frequencies or one or more ranges of frequencies of the first resultant signal can indicate a higher concentration of an analyte of interest in a first fluid medium than a second resultant signal having a lesser amplitude loss at the same one or more specific frequencies or the same one or more ranges of frequencies of the second signal produced after transmitting an identical radiofrequency transmission signal through the same amount of fluid medium.

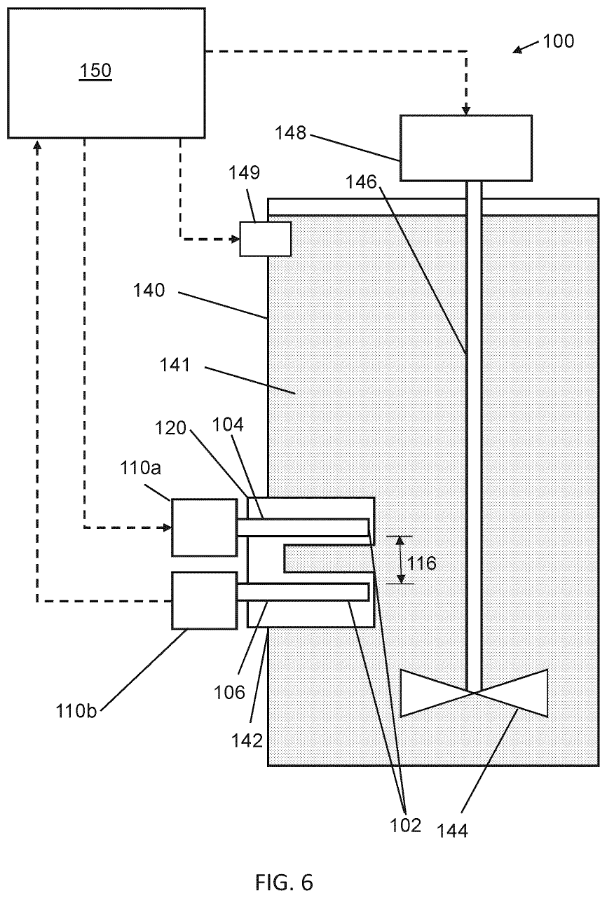

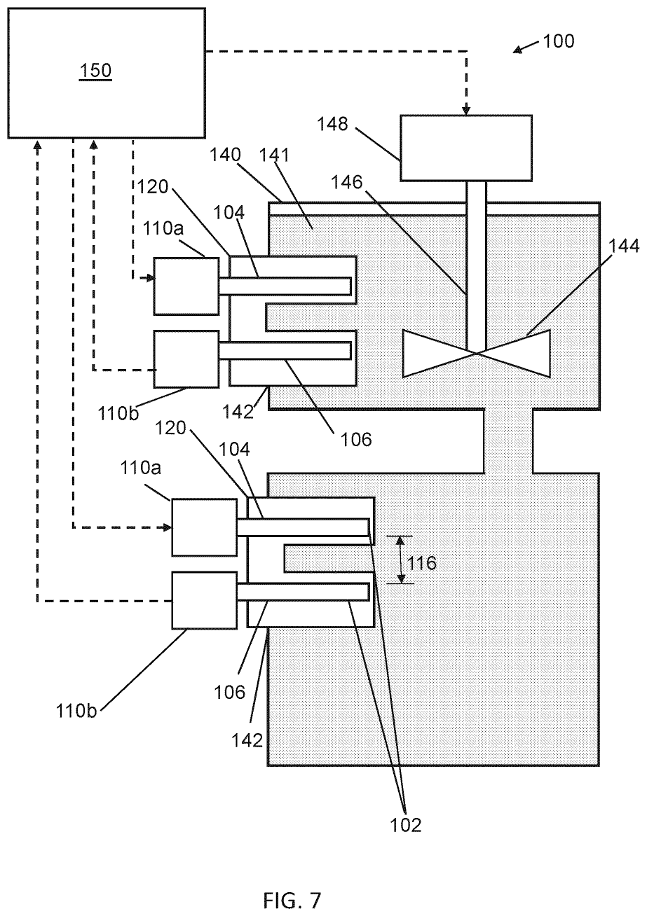

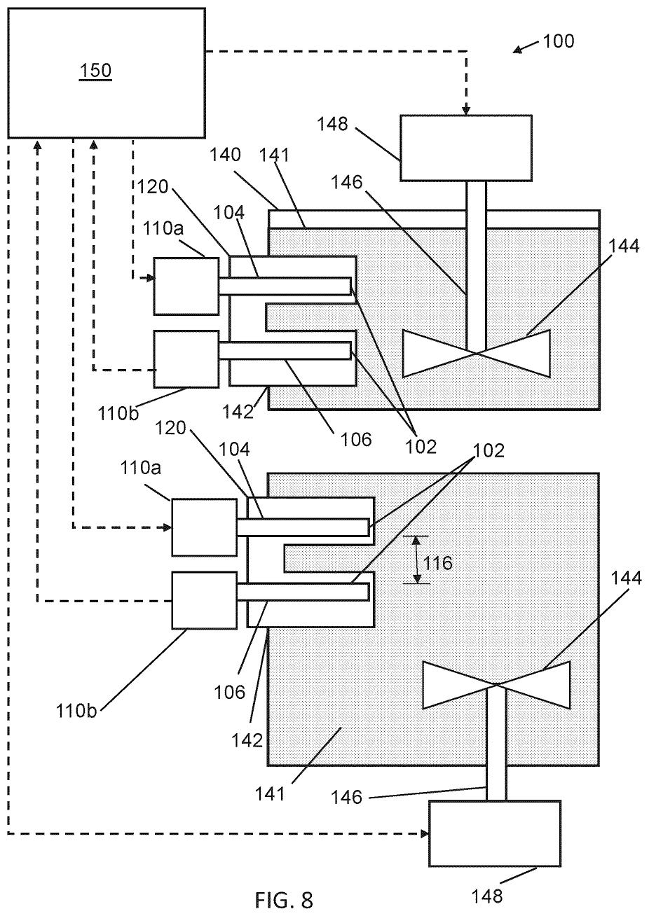

In some cases, a radiofrequency signal transmitted from an RF transmitter 104 of an RF sensor through a fluid medium (e.g., a radiofrequency transmission signal) and received by an RF receiver 106 of the RF sensor 102 can be transmitted as a resultant signal to a computer system controller 150, for example, via a wired connection or a wireless connection (dashed lines in FIG. 6, FIG. 7, and FIG. 8 represent a wired or wireless connection by which digital or analog data, such as the data of a radiofrequency signal, can be transmitted). In some cases, a resultant signal can comprise the radiofrequency signal data received at the RF receiver 106 of the RF sensor after the radiofrequency signal is transmitted from the RF transmitter 104 through the fluid medium 141, which may include changes (e.g., alterations) in the amplitude and phase modulation of the signal (e.g., the pattern of the signal) imparted by passing the radiofrequency signal through the fluid medium. In some cases, RF sensor 102 can comprise a communication element 110b configured to transmit the resultant signal to the computer system controller 150 (e.g., via a wireless or wired connection).

A computer system controller 150 can be configured to analyze a resultant signal (e.g., comprising changes in a radiofrequency signal resulting from the transmission of the radiofrequency signal through a fluid medium 141 comprising one or more analytes of interest). In some cases, a resultant signal transmitted to a computer system controller 150 of a system or method described herein can be compared to one or more radiofrequency signals stored in a memory of the computer system controller and/or to one or more resultant signals received at the computer system controller 150 (e.g., from an RF sensor 102 measuring the presence, absence, or concentration of one or more analytes of interest in a second region or chamber of the same bioprocess container or in a second bioprocess container, for example, wherein the second region, chamber, or bioprocess container is has a known concentration of the one or more analytes). In some cases, a computer system controller 150 can be calibrated and/or trained (e.g., in the case of analysis using computer learning systems and methods) to determine a relative or absolute (e.g., numerical) amount of the one or more analytes of interest, for instance, based completely or in part on one or more changes or alterations in the amplitude or phase modulation of a radiofrequency signal at one or more specific frequencies or one or more ranges of frequencies of the radiofrequency signal. In some cases, a computer system controller 150 can be configured to compare the determined relative absolute amount of the one or more analytes of interest to a threshold value (e.g., concentration) or an absolute or relative tolerance for the amount of the one or more analytes in the fluid medium 141, for instance, to determine compliance with acceptable or desired bioreactor condition parameters. In some cases, an increase (or in other cases, a decrease) in one or more analytes in a fluid medium of a bioprocess container can be indicative of a quantity or change in quantity or health status of cells within the bioprocess container, a level or change of viability of a population of cells in the bioprocess container, and/or a level or change in function of a population of cells in the bioprocess container (e.g., production of a biological product by the population of cells).

In some embodiments, a computer system controller 150 can be configured to transmit a driver signal 164 to an RF transmitter 104 of an RF sensor 102, e.g., for transmission by the RF transmitter 104 to through a fluid medium 141 of a bioprocess container 140 as a radiofrequency transmission signal. In some implementations, a driver signal 164 can be used to determine one or more changes or alterations in an amplitude and or one or more changes or alterations in a phase modulation of a resultant signal. For example, all or a portion of the driver signal 164 can be compared to the corresponding portion(s) of a resultant signal received by the computer system controller 150 from the RF receiver 106 to determine one or more changes or alterations in amplitude or phase modulation present in the resultant signal. In some embodiments, the one or more changes or alterations in amplitude or phase modulation present in the resultant signal may be caused by interactions between one or more analytes in the fluid medium 141 and a radiofrequency transmission signal upon which the resultant signal is based. In some embodiments, a computer system controller 150 can comprise an input/output device 1440 (which may comprise a wired or wireless transmitter) configured to transmit the driver signal 164 to the RF transmitter 104 of an RF sensor 102. In some implementations, a computer system controller 150 can be configured to transmit one or more driver signals 164 (e.g., a plurality of identical driver signals or a plurality of different driver signals) to a plurality of RF sensors 102. For example, a computer system controller 150 can be configured to cause a plurality of the RF sensors 102 to transmit radiofrequency signals through fluid medium in different portions of the same bioprocess container. In some embodiments, a computer system controller 150 can be configured to cause a plurality of RF sensors 102 to transmit radiofrequency signals through fluid media of different bioprocess containers. In some implementations, a computer system controller 150 can transmit a plurality of identical driver signals or a plurality of different driver signals to a plurality of RF sensors 102. In some implementations, the frequency range, amplitude, or phase modulation of a driver signal can be selected to interrogate a fluid medium for a set of one or more specific analytes of interest. In some implementations, for example, where modulation of a radiofrequency signal amplitude and/or phase is known to occur in one or more specific frequencies or frequency ranges when a broad spectrum radiofrequency signal interacts with a set of one or more specific analytes of interest, a driver signal 164 including the one or more specific frequencies or frequency ranges can be transmitted to one or more RF transmitters 106. In some implementations, the driver signal 164 can comprise, consist of, or consist essentially of a truncated or non-continuous frequency sweep comprising the frequency values or ranges at which the modulation is known to occur

In some embodiments, an input/output device 1440 of a computer system controller 150 can comprise a receiver, which may be configured to receive a signal from one or more RF receivers 106. For example, the signal can include an analog or digitally formatted resultant signal comprising data reflecting a radiofrequency signal pattern. Instructions stored in a memory 1420 of a computer system controller 150 can be configured, when executed by a processor 1410 of the computer system controller 150 to analyze the resultant signal. In some cases, analyzing the resultant signal can comprise determining one or more differences (e.g., changes or alterations) in the resultant signal or a portion or aspect thereof (e.g., an amplitude change or phase modulation of all or a portion of the resultant signal with respect to a reference signal). In some implementations, determining one or more differences in the resultant signal can be used to determine the presence or absence of such a pattern in a resultant signal received from an RF receiver 106. Determining one or more differences in the resultant signal can be used to determine the characteristic aspects of a radiofrequency signal pattern specific to the presence, absence, or amount of one or more analytes of interest in a fluid medium 141. Determination of characteristic aspects of a radiofrequency signal pattern that change specifically or reproducibly when a radiofrequency signal interacts with one or more analytes of interest in a fluid medium 141 can be useful in the creation of a driver signal 164.

In some cases, a computer system controller 150 of a radiofrequency bioreactor sensor system 100 can be used to identify a pattern of a radiofrequency signal indicative of the presence, absence, or relative or absolute concentration of one or more analytes of interest in a fluid medium 141 of a bioprocess container 140, for instance, using machine learning. In some cases, a training set of data can be used to train a machine learning module of the computer system 150, for example, to identify a pattern of characteristics of a radiofrequency signal (e.g., signal amplitude and/or phase modulation). For example, one or more specific frequencies or one or more ranges of frequencies of the signal, that are indicative of interaction with one or more specific analytes in the fluid medium 141. Computer system controller 150 can comprise a vector network analyzer (VNA). In some cases, the computer system controller 150 or additional computer or computer module can be used to perform analysis of radiofrequency signals and radiofrequency signal patterns (e.g., resultant signals), for instance, utilizing a trained machine learning algorithm. In some cases, the additional computer or computer module (e.g., controller) can be used to control VNA with respect to what frequencies should be used. In some cases, the machine learning module may employ one or more supervised machine learning algorithms for identification of patterns of characteristics of a radiofrequency signal specific to the presence, absence, or concentration of one or more analytes in the fluid medium. In some cases, the machine learning module may employ one or more unsupervised machine learning algorithms for identification of patterns of characteristics of a radiofrequency signal specific to the presence, absence, or concentration of one or more analytes in the fluid medium. Training the machine learning module to accurately identify patterns of characteristics of a radiofrequency signal specific to the presence, absence, or concentration of one or more analytes in the fluid medium can comprise comparing predictions made by the machine learning module regarding the presence, absence, or concentration of one or more analytes to patterns identified as specifically reflecting a presence, absence, or specific concentration of the one or more analytes.

An RF sensor system 100 can comprise a radiofrequency sensor introducer 120, for instance, to allow for use of one or more RF sensors 102 within a bioprocess container's interior. FIG. 6, FIG. 7, and FIG. 8 show embodiments of RF sensor systems 100 in which RF sensors 102 (e.g., comprising one or more RF transmitters 104 and one or more RF receivers 106) are provided to an interior of a bioprocess container using an introducer 120. An introducer 120 can comprise one or more introducer cavities 124 and, optionally, one or more introducer sheaths 126a, 126b. As exemplified in FIGS. 6-8, introducer 120 can be introduced into an interior of a bioprocess container 140 when inserted through a port 142 of the bioprocess container 140. Introducer 120 can be coupled to port 142 or a port adapter 132 of bioprocess container 140 to form a watertight connection. Once physically disposed within the bioprocess container 140, a fluid 141 within bioprocess container 140 can be disposed in between an RF transmitter 104 and an RF receiver 106 installed in the introducer 120. In this way, an RF signal can be used to analyze one or more analytes of interest in a fluid of a bioprocess container 140, for example, wherein it is desired or advantageous that a separation distance between the RF transmitter 104 and the RF receiver 106 is small relative to one or more dimensions of the bioprocess container 140. In some embodiments, a smaller separation distance between the RF transmitter 104 and the RF receiver 106 than would be possible if the RF sensors were placed outside of the bioprocess container 140 may result in more accurate or more easily distinguished measurements of analytes of interest in the fluid 141. Accordingly, introducer 120 can be used to position one or more RF sensors 102 (e.g., one or more RF transmitters 104 and/or one or more RF receivers 106) inside of a bioprocess container 140, e.g., to improve accuracy or signal-to-noise ratio in analysis of one or more analytes of interest in the fluid with an RF signal.

As shown in FIGS. 6-8, an RF transmitter 104 can comprise a communication element 110a and an RF receiver 106 can comprise a communication element 110b, e.g., for transmitting data to or receiving data from a computer system 150 (e.g., as indicated by dashed lines in FIGS. 6-8). As described herein, a driver signal can be transmitted from the computer system 150 (e.g., via a wired or wireless connection) to the RF transmitter 104, which can use the driver signal to produce an RF signal and transmit the RF signal into the fluid 141 of the bioprocess container. An RF signal can be received by the RF receiver 106 and transmitted back to the computer system 150 (e.g., via a wired or wireless connection), for example, for processing in analysis of one or more analytes of interest in the fluid 141.

A computer system controller 150 can be configured to operate one or more pieces of equipment of a radiofrequency sensor system 100, for example, based at least in part on an analysis of one or more resultant signals. In some embodiments, a computer system controller 150 can be configured to operate a motor 148 of a mixer to turn a drive shaft 146 of the mixer such that an impeller 144 coupled to the drive shaft 146 turns in response to the turning of the drive shaft 146. For example, analysis of resultant signals received from a plurality of RF sensors 102 configured to interrogate (e.g., to pass a radiofrequency transmission signal through) fluid medium in different regions or chambers of a bioprocess system reflecting different concentrations of a nutrient such as glucose may indicate insufficient mixing in the bioprocess container (e.g., as determined by glucose concentration measured using a resultant signal at one or more RF sensors 102 being below a threshold value), in which case the computer system controller 150 configured to operate the mixer may send a signal to motor 148 to turn drive shaft 146 and impeller 144. In some embodiments, a computer system controller 150 can be configured to operate a reagent handling system 149. For example, analysis of one or more resultant signals received from one or more RF sensors 102 configured to interrogate a fluid medium 141 (e.g., cell culture medium) of a bioprocess container 140 (e.g., bioreactor) reflecting a high concentration of an analyte of interest (e.g., a concentration of lactate or one or more amino acids higher than a target threshold value) performed by the computer system controller 150 can cause the computer system controller 150 to operate a reagent handling system 149 to add fluid medium (e.g. cell culture medium) and/or other nutrients to the bioprocess container, for example, to improve or maintain a phenotypic state of a population of cells in the bioprocess container (e.g., by adding necessary nutrients to the culture that may be low or missing and/or by diluting potentially deleterious reagents present in the culture). In some embodiments, analysis of one or more resultant signals received from one or more RF sensors 102 reflecting a concentration of glucose lower than a target threshold concentration can cause computer system controller 150 to operate a reagent handling system 149 to add glucose (e.g., “feed”) to the fluid medium of the bioprocess container, for example, to return glucose concentration to, above, or between one or more target threshold concentrations. In some cases, a computer system controller 150 can be configured to operate a mixer motor mount (or a linkage system of a mixer motor mount) to raise or lower a mixer in the bioprocess container, for example, in cases where the computer system controller 150 is operating the reagent handling system to add fluid medium to the bioprocess container.

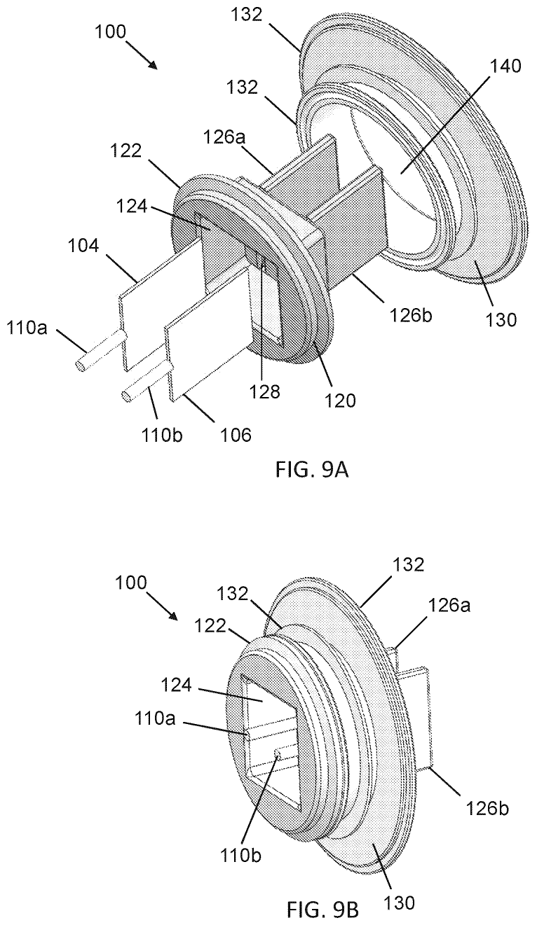

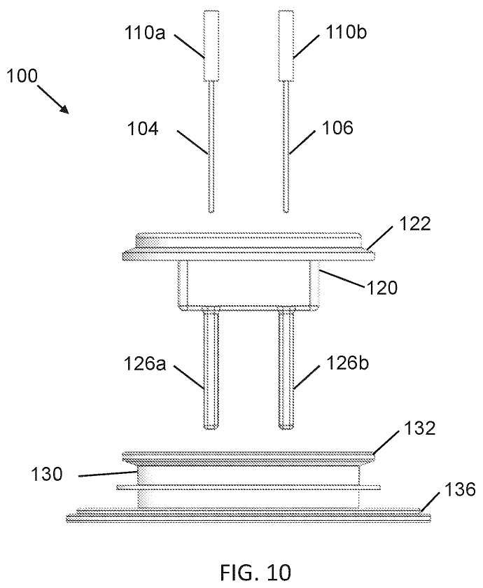

FIGS. 9A-10 illustrate an introducer 120 that can maintain a position and orientation of the RF transmitter 104 relative to the RF receiver 106 of the RF sensor 102. In some embodiments, an introducer 120 can comprise a rigid or semi-rigid material, such as a hard plastic. In some embodiments, all or a portion of an introducer 120 can be sterilizable (e.g., autoclavable), for example, to allow reuse. All or a portion of introducer 120 (e.g., RF transmitter sheath 126a and/or RF receiver sheath 126b) can be made of a radio transparent material, such as Biomed Clear SLA. For example, all or a portion of introducer 120, including all or a portion of RF transmitter sheath 126a and/or all or a portion of RF receiver sheath 126b can be made of a radio transparent material, such as Biomed Clear SLA. In some cases, an introducer 120 can be made of single-use material. In some cases, an introducer 120 can be disposable (e.g., single-use). In some cases, RF transmitter 104, RF receiver 106, and/or communication element 110a or 110b can be disposable (e.g., single-use). In some implementations, an entire assembly comprising introducer 120, RF transmitter 104, and RF receiver 106 can be implemented as a single-use component. In some embodiments, the RF transmitter 104 and RF receiver 106 can be releasably attachable to the body of the introducer 120. In some cases, introducer 120 can comprise one or more sensor sheaths 126 configured to receive an RF transmitter 104 or an RF receiver 106. For example, FIG. 9A shows an introducer comprising an RF transmitter sheath 126a, configured to slidably receive RF transmitter 104 and an RF receiver sheath 126b configured to slidably receive RF receiver 106 (e.g., as shown in FIG. 9B). For example, the transmitter sheath 126a can include a cavity 128 having a similar shape and/or dimensions as the RF transmitter 104. This configuration can assist with maintaining a position of the RF transmitter 104 along the transmitter sheath 126b. In addition, the receiver sheath 126b can include a cavity having a similar shape and/or dimensions as the RF receiver 126. Such a configuration can assist with maintaining a position of the RF receiver 106 along the receiver sheath 126a. This can allow for efficient and accurate alignment between the RF transmitter 104 and RF receiver 106. For example, an RF transmitter sheath 126a and/or an RF receiver sheath 126b can have a shape, a depth, or an angle configured to maintain an RF transmitter 104 and/or an RF receiver 106 at preferred alignment position(s). In some embodiments, a preferred alignment position can comprise parallel arrangement of a first plane in which an RF transmitter 104 is entirely contained and a second plane in which an RF receiver 106 is entirely contained, wherein a straight line connecting the center of the RF transmitter 104 and the center of the RF receiver 106 is perpendicular to both the first plane and the second plane.

FIG. 9B illustrates an introducer 120 that can be configured such that the RF transmitter 104 and RF receiver 106 can be passed through the body of the introducer 120 via a recess 124 of introducer 120 and into a cavity 128 of the respective RF transmitter sheath 126a or RF receiver sheath 126b. In some cases, a sensor sheath 126 of an introducer 120 (e.g., an RF transmitter sheath 126a and/or an RF receiver sheath 126b) can be configured to prevent an RF transmitter 104 or RF receiver 106 from contacting fluid medium 141 within bioprocess container 140. In some cases, engaging the RF transmitter 104 and RF receiver 106 with the respective RF transmitter sheath 126a and RF receiver sheath 126b can position the RF transmitter 104 and RF receiver 106 in the interior of a bioprocess container 140 with a space of known distance 116 and orientation between them. This can position the RF sensor 102 components advantageously when fluid medium 141 is present in the bioprocess container 140, for example, wherein the fluid medium is disposed between the RF transmitter antenna 112 and the RF receiver antenna 114. In some cases, the rigid or semi-rigid sheath(s) of the introducer 120 can maintain the relative position and orientation (and sensor separation distance) of the RF transmitter 104 with respect to the RF receiver 106. In some cases, the sheath(s) of introducer 120 can be rigidly or semi-rigidly coupled to the body of the introducer. For example, the sheath(s) of introducer 120 can be rigidly or semi-rigidly coupled to a recess of the introducer. In some cases, an introducer can use a clip, door, or locking mechanism to ensure that RF transmitter 104 and RF receiver 106 remain in registry with the introducer 120 (e.g., with the respective sheaths of the introducer).

In some embodiments, introducer 120 can advantageously prevent contact between the RF transmitter 104 and/or the RF receiver 106 and the fluid medium 141 of bioprocess container 140. For example, RF transmitter sheath 126a can comprise a physical barrier between RF transmitter 104 and the fluid medium 141. In some implementations, RF receiver sheath 126b can comprise a physical barrier between RF receiver 106 and the fluid medium 141. In some cases, RF transmitters 104 and RF receivers 106 may not be easily cleaned of residual fluid medium, and deposits of the fluid medium may remain on the RF sensor 102 components, which may degrade the RF sensor 102 components and/or interfere with signal transmission or receipt. Physically isolating the RF transmitter 104 and the RF receiver 106 from the fluid medium 141 can also allow quick removal of the RF transmitter 104 and RF receiver 106 from the bioprocess container 140. Physically isolating the RF transmitter 104 and the RF receiver 106 from the contents of the bioprocess container 140 can be important for maintaining sterility of the contents of the bioprocess container 140 and/or avoiding corrosion or degradation of the RF transmitter 104, the RF receiver 106, or a portion thereof. In some cases, the one or more sensor sheaths 126 of an introducer 120 can allow the RF transmitter 104 and RF receiver 106 to be removed from the introducer 120 without unscaling the bioprocess container 140. In some cases, an introducer can form a sealed container when coupled to the bioprocess container 140.

In some embodiments, an introducer 120 can comprise an introducer coupling interface 122, which can be configured to interface with (e.g., couple to) a port 142 or port adapter 130 of the bioprocess container 140 (see, FIG. 9A, FIG. 9B, FIG. 10). Port 142 can be an opening in the bioprocess container. In some implementations, a port adapter 130 can be useful for maintaining the shape and orientation of port 142, for example, when bioprocess container 140 is a flexible bag. In some embodiments, a port adapter 130 can be disposable (e.g., configured for single use by selection of port adapter 130 material). In some cases, an introducer coupling interface 122 can comprise threading for interfacing with the port 142 or port adapter 130. In some cases, attaching introducer coupling interface 122 with port 142 or port adapter 130 forms a watertight seal, preventing leakage of fluid medium 141 from the bioprocess container 140. In some cases, all or a portion of introducer 120 can comprise cladding or electrical shielding, for example, to reduce or prevent interference from outside electrical signals on transmission or receipt of a radiofrequency signal described herein. In some cases, an introducer can comprise a cage structure partially or completely surrounding the RF transmitter 104, the RF receiver 106, and/or the sheath(s) of the introducer 120. For example, this can reduce or eliminate interference from outside electrical signals while allowing fluid medium to pass through the cage to the space between the RF transmitter 104 and the RF receiver 106.

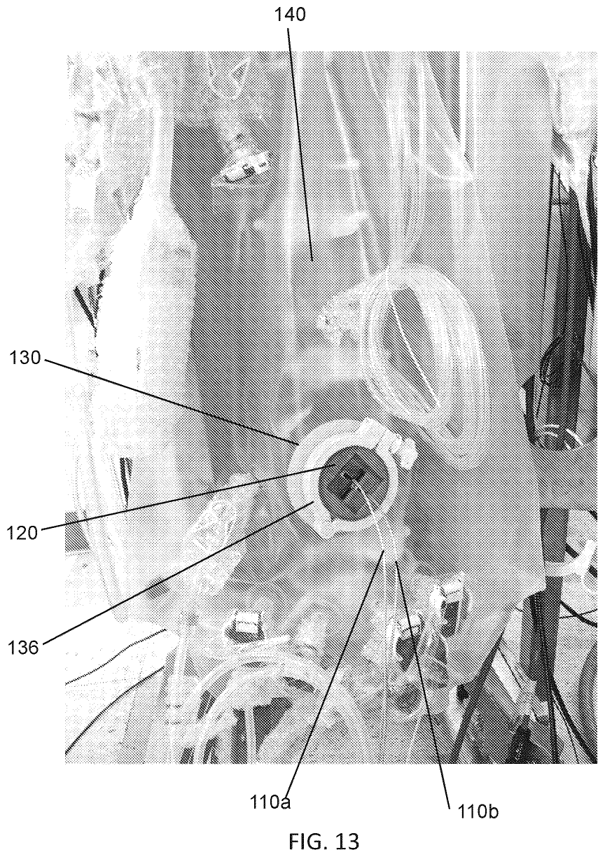

FIGS. 11-13 illustrate embodiments of the port adapter 130 coupled to a bioprocess container 140, which can include one or more flexible walls. For example, the port adapter 130 can make securing an introducer 120 directly to a port 142 of the bioprocess container 140 less difficult. In some cases, inclusion of a port adapter 130 in a system, device, or method described herein can improve the strength and/or water-tightness of the interface between the introducer 120 and/or reduce the risk of damaging (e.g., tearing or compromising) a wall of the bioprocess container. Some embodiments of the port adapter 130 can comprise a port adapter bioprocess container interface 134, which can be configured to form a watertight seal with a port 142 of a bioprocess container 140. A port adapter 130 can comprise a port adapter coupling interface 132, which can be configured to couple with an introducer 120 (e.g., by directly coupling with introducer coupling interface 122 of the introducer 120). In some cases, a port adapter coupling interface 132 can form a watertight seal with introducer 120 when the two are coupled together. In some cases, a port adapter coupling interface 132 can threadedly engage the introducer 120. In some cases, a port adapter coupling interface 132 can form a snap-fit with introducer 120. In some cases, a port adapter coupling interface 132 can comprise a port adapter fastener 136, for example, as shown in FIGS. 11-13, which can be used to secure the introducer 120 to the port adapter 130. In some embodiments, a port adapter fastener 136 can comprise a clamp, a clip, or a threaded adapter. In some embodiments, a port adapter fastener 136 or a port adapter 130 can comprise a gasket, for example, to ensure water-tightness when the bioprocess container is in use. In some embodiments, a bioprocess container 140 comprising a rigid container can be implemented with a port adapter 130 and, optionally, a bioprocess container interface 132.

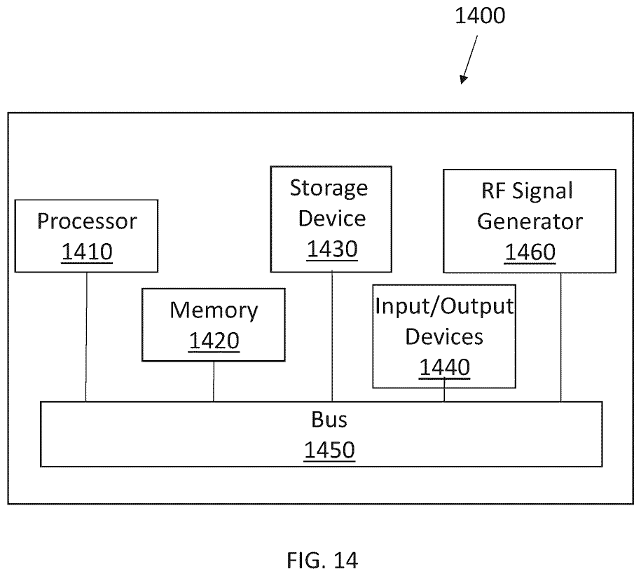

FIG. 14 illustrates a block diagram depicting an example of a computing system 150 (e.g., controller 150) consistent with implementations of the current subject matter. The computing system 150 can be used to implement a radiofrequency bioreactor sensor system 100 and/or any component therein. For example, the computing system 150 can implement user equipment, a personal computer, or a mobile device.

As shown in FIG. 14, the computing system 150 can include a processor 1410, a memory 1420, a storage device 1430, an input/output device 1440, an RF signal generator 1460, and/or an RF signal receiver 1470. The processor 1410, the memory 1420, the storage device 1430, the input/output device 1440, the RF signal generator 1460, and the RF signal receiver 1470 can be interconnected via a system bus 1450. The processor 1410 is capable of processing instructions for execution within the computing system 1400. Such executed instructions can implement one or more components of, for example, the radiofrequency sensor system 100 for calculating a presence, absence, or concentration of one or more analytes in a fluid medium of a bioprocess container and determining whether an analyte concentration threshold is satisfied. In some example embodiments, the processor 1410 can be a single-threaded processor. Alternately, the processor 1410 can be a multi-threaded processor. The processor 1410 is capable of processing instructions stored in the memory 1420 and/or on the storage device 1430 to display graphical information for a user interface provided via the input/output device 1440.

The memory 1420 can be a non-transitory computer-readable medium that stores information within the computing system 150. The memory 1420 can store data structures representing configuration object databases, for example. The storage device 1430 can be capable of providing persistent storage for the computing system 150. The storage device 1430 can be a floppy disk device, a hard disk device, an optical disk device, or a tape device, or other suitable persistent storage means. The input/output device 1440 provides input/output operations for the computing system 150. In some example embodiments, the input/output device 1440 includes a physical or virtual keyboard and/or pointing device. In various implementations, the input/output device 1440 includes a display unit for displaying graphical user interfaces. The display unit can be a touch activated screen that displays and facilitates user input/output operations.

According to some example embodiments, the input/output device 1440 can provide input/output operations for a network device. For example, the input/output device 1440 can include Ethernet ports or other networking ports to communicate with one or more wired and/or wireless networks (e.g., a local area network (LAN), a wide area network (WAN), the Internet, a public land mobile network (PLMN), and/or the like). Other communication protocols can include analog, digital and/or other communication signals.

In some example embodiments, the computing system 150 can be used to execute various interactive computer software applications that can be used for organization, analysis, and/or storage of data in various formats. Alternatively, the computing system 150 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing items and/or functionalities. Upon activation within the applications, the functionalities can be used to generate the user interface provided via the input/output device 1440. The user interface can be generated and presented to a user by the computing system 150 (e.g., on a computer screen monitor, etc.).

In some embodiments, a memory 1420 of computing system 150 (e.g., controller 150) can comprise instructions stored thereupon that, when executed by processor 1410 of the computer system 150, can operate one or more components of a system or device described herein and/or can cause the system or device to perform one or more method steps described herein.

In some embodiments, a computing system 150 can comprise an RF signal generator 1460. An RF signal generator 1460 can be a computer module configured to produce a driver signal 164, which can be transmitted to an RF transmitter 104, for example, via a wired or wireless communication connection 110. In some embodiments, a computer system 150 can comprise an RF signal receiver 1470. An RF signal receiver 1470 can be a computer module configured to receive, reformat, and/or analyze an RF signal or data resulting from an RF signal (e.g., a resultant signal) communicated to the RF signal receiver 1470, for example, via a wired or wireless communication connection 110.

Methods for measuring one or more analytes in a fluid medium (e.g., a cell culture medium or in-process fluid) can comprise the use of one or more systems or devices described herein. In some embodiments, a method described herein can comprise measuring an analyte of interest in a fluid medium using a radiofrequency signal. The method can comprise transmitting a radiofrequency signal through a fluid medium 141 of a bioreactor 140. Some methods described herein can comprise determining a presence, absence, or concentration of an analyte of interest based at least in part on a radiofrequency signal. In some embodiments, the method can comprise operating one or more bioreactor components based on the determined presence, absence, or concentration of the analyte of interest.

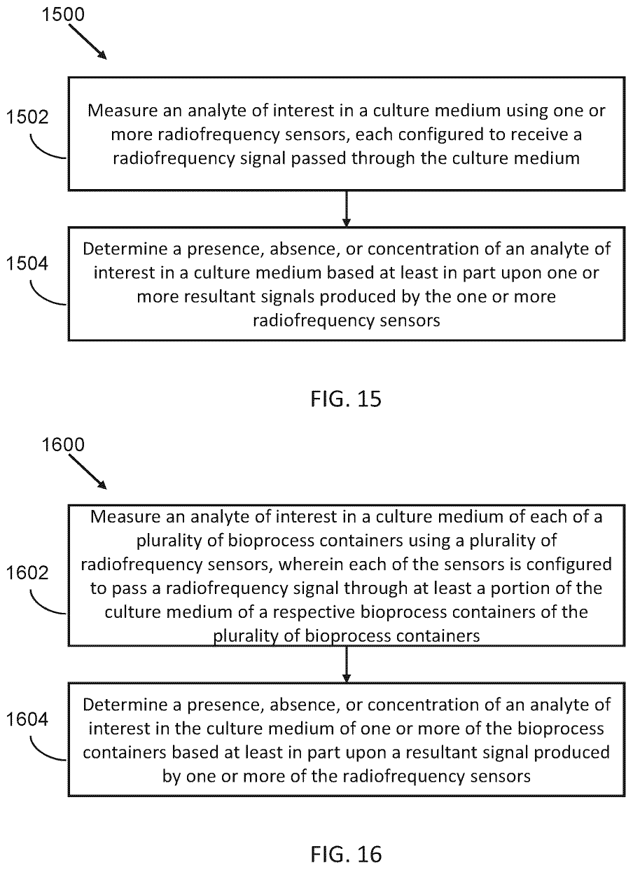

FIG. 15 illustrates steps of a method 1500 can comprise a step 1502, which can comprise measuring an analyte of interest in a fluid medium using one or more radiofrequency sensors, each configured to receive a radiofrequency signal passed through the fluid medium, and a step 1504, which can comprise determining a presence, absence, or concentration of an analyte of interest in a fluid medium based at least in part upon one or more resultant signals produced by the one or more radiofrequency sensors.

FIG. 16 illustrates steps of a method 1600 can comprise a step 1602, which can comprise measuring an analyte of interest in a fluid medium of each of a plurality of bioprocess containers using a plurality of radiofrequency sensors, wherein each of the sensors is configured to pass a radiofrequency signal through at least a portion of the fluid medium of a respective bioprocess container of the plurality of bioprocess containers, and a step 1604, which can comprise determining a presence, absence, or concentration of an analyte of interest in the fluid medium of one or more of the bioprocess containers based at least in part upon a resultant signal produced by one or more of the radiofrequency sensors.

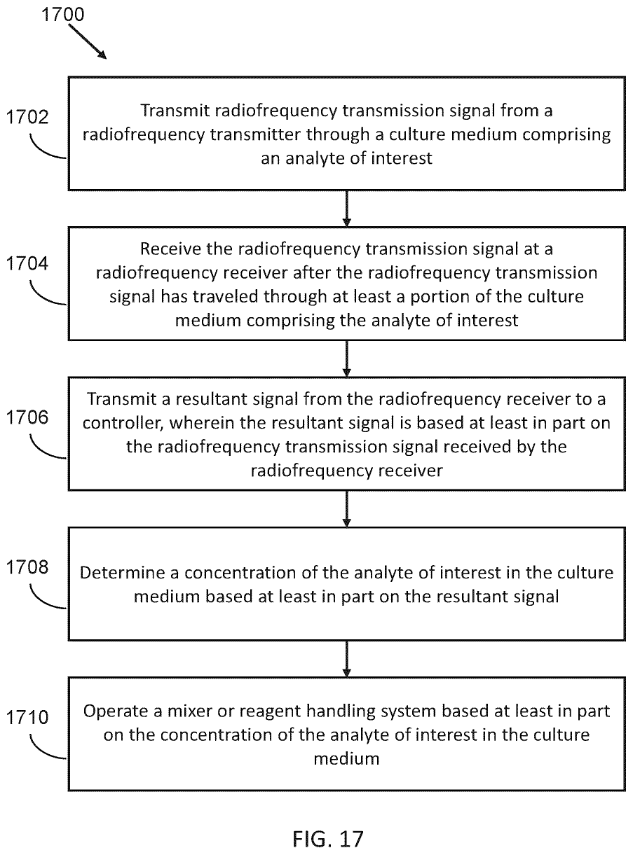

FIG. 17 illustrates steps of a method 1700, which can comprise a step 1702, which can comprise transmitting a radiofrequency transmission signal from a radiofrequency transmitter through a fluid medium comprising an analyte of interest, a step 1704, which can comprise receiving the radiofrequency transmission signal at a radiofrequency receiver after the radiofrequency transmission signal has traveled through at least a portion of the fluid medium comprising the analyte of interest, a step 1706, which can comprise transmitting a resultant signal form the radiofrequency receiver to a controller, wherein the resultant signal is based at least in part on the radiofrequency transmission signal received by the radiofrequency receiver, a step 1708, which can comprise determining a concentration of the analyte of interest in the fluid medium based at least in part on the resultant signal, and/or a step 1710, which can comprise operating a mixer or reagent handling system based at least in part on the concentration of the analyte of interest in the fluid medium.

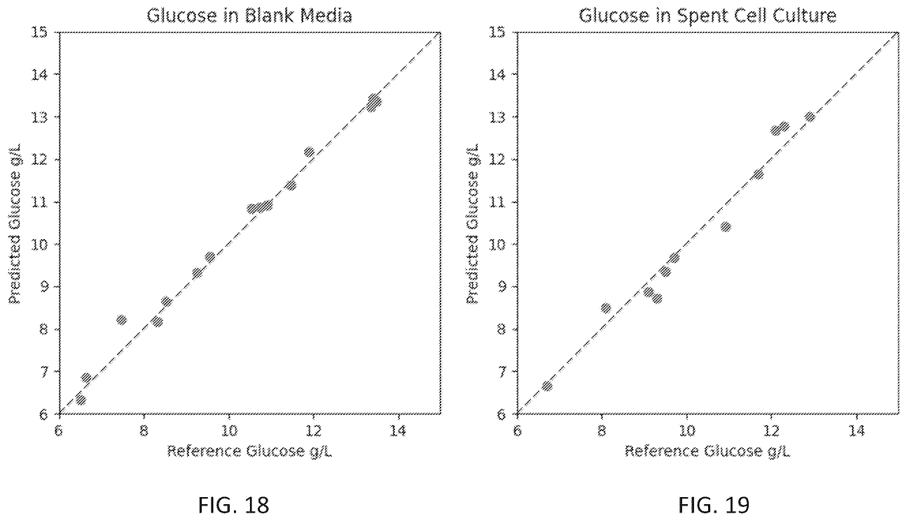

FIGS. 18 and 19 show fidelity of predicted radiofrequency glucose (RF glucose) concentrations (“Test Set”) in a fluid medium of a bioreactor plotted against the known concentrations of glucose in the fluid medium of the bioreactor (“Training Set”). FIG. 18 shows predicted values for glucose concentration in fresh medium (dotted line) using radiofrequency detection, as described herein, versus known reference values of glucose concentration in fresh medium (circular dots). FIG. 19 shows predicted values for glucose concentration in spent (e.g., conditioned) medium (dotted line) using radiofrequency detection, as described herein, versus known reference values of glucose concentration in spent (e.g., conditioned) medium (circular dots). FIGS. 18 and 19 show good correlation of predicted concentrations determined using radiofrequency measurement and measured reference values.

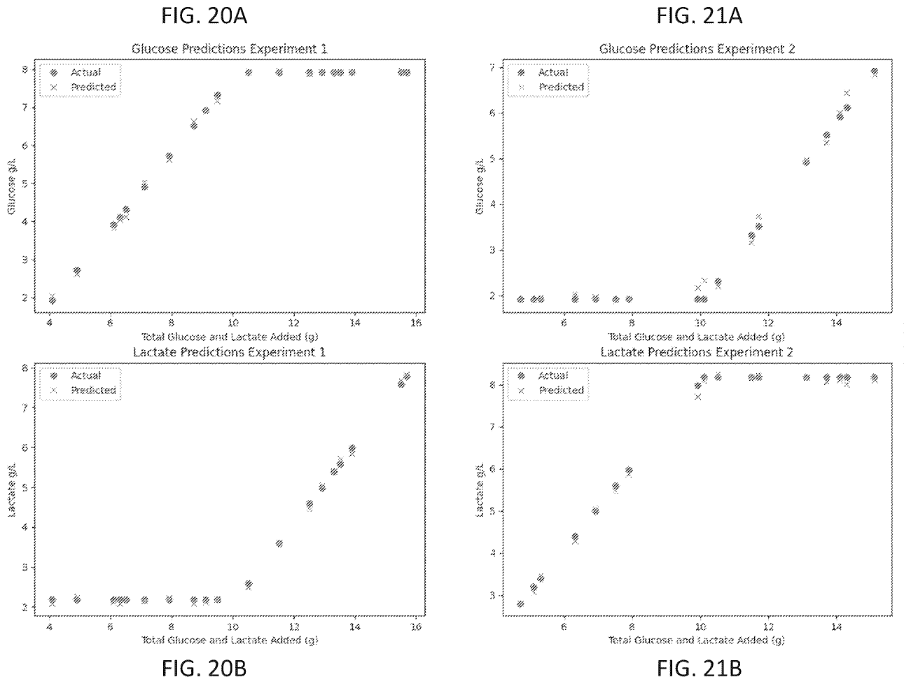

FIGS. 20A and 21A show training and test set data related to machine learning training for glucose concentration determination in a fluid medium (Experiment 1 and Experiment 2, respectively). FIGS. 20B and 21B show training and test set data related to machine learning training for lactate concentration determination in a fluid medium (Experiment 1 and Experiment 2, respectively). In each of FIGS. 20A-21B, filled circles represent measured data, and “x” data points indicate predicted values. In each of FIGS. 20A-21B, the x-axis represents total glucose and lactate added in grams (g). Marked x-axis values in FIGS. 20A-20B are 4 g, 6 g, 8 g, 10 g, 12 g, 14 g, and 16 g total glucose and lactate added, from left to right. Marked x-axis values in FIGS. 21A-21B are 6 g, 8 g, 10 g, 12 g, and 14 g, from left to right total glucose and lactate added. Marked y-axis values in FIG. 20A are 2 g, 3 g, 4 g, 5 g, 6 g, 7 g, and 8 g of glucose (grams/liter), from bottom to top. Marked y-axis values in FIG. 20B are 2 g, 3 g, 4 g, 5 g, 6 g, 7 g, and 8 g of lactate (grams/liter), from bottom to top. Marked y-axis values in FIG. 21A are 2 g, 3 g, 4 g, 5 g, 6 g, and 7 g of glucose (grams/liter), from bottom to top. Marked y-axis values in FIG. 21B are 3 g, 4 g, 5 g, 6 g, 7 g, and 8 g of lactate (grams/liter), from bottom to top.

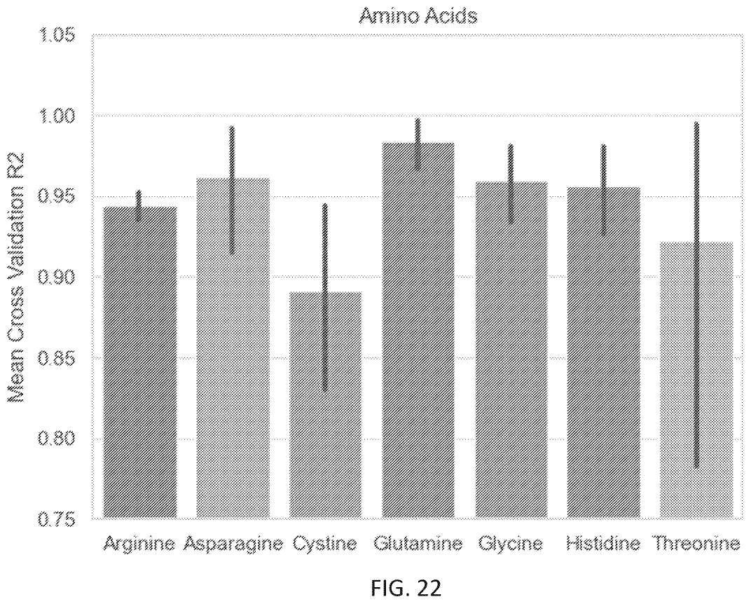

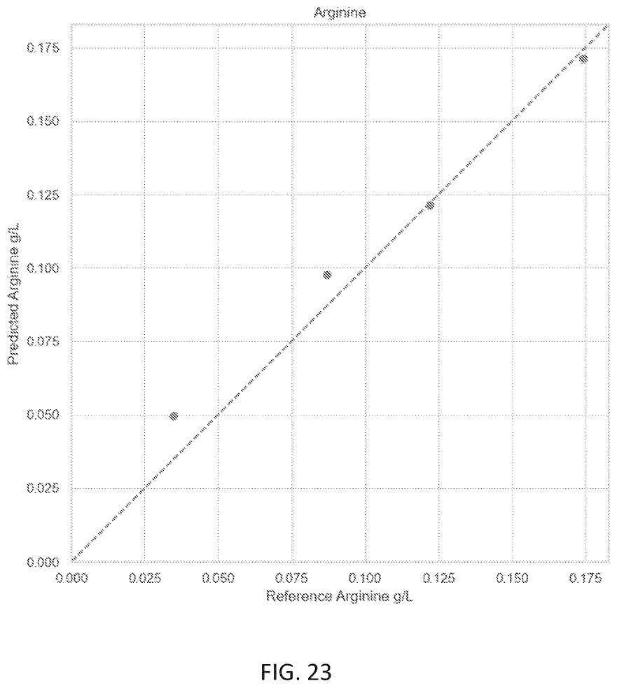

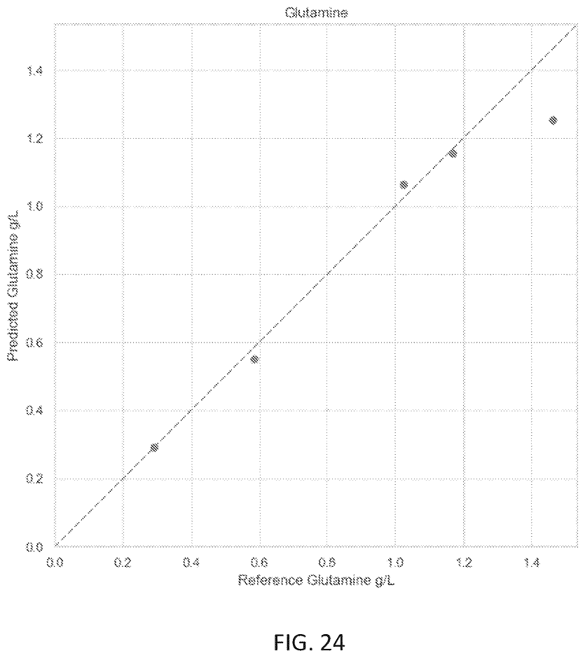

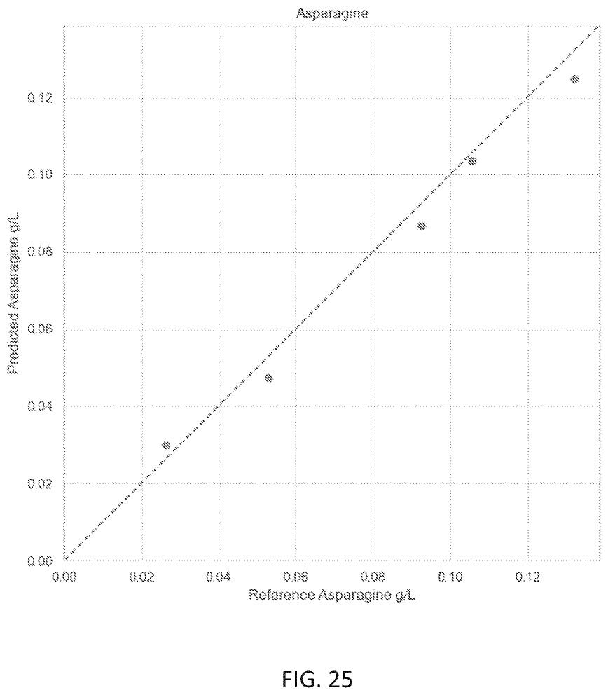

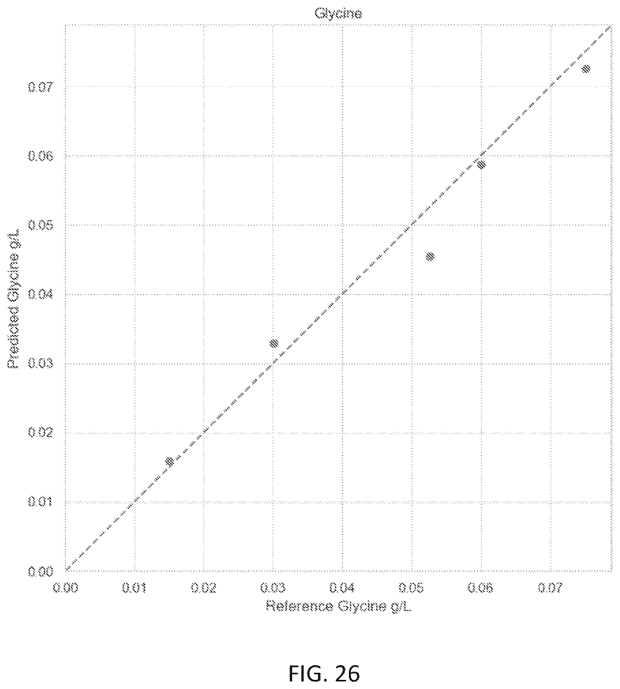

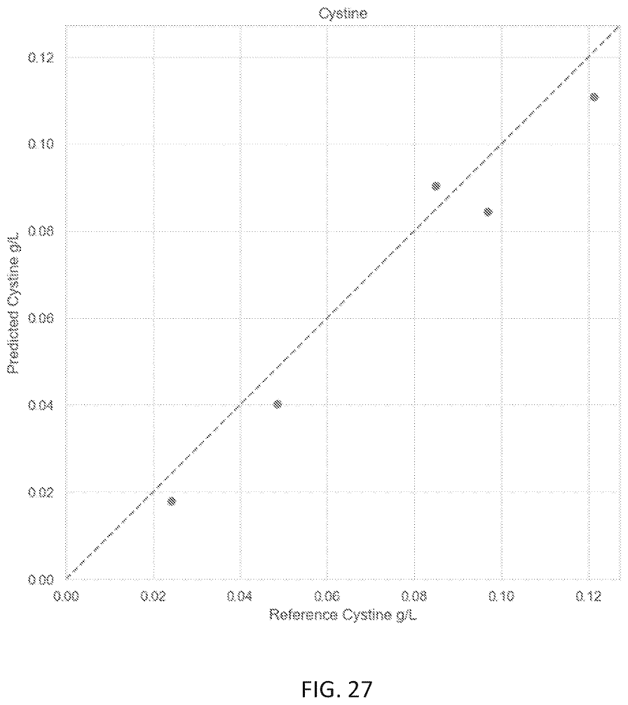

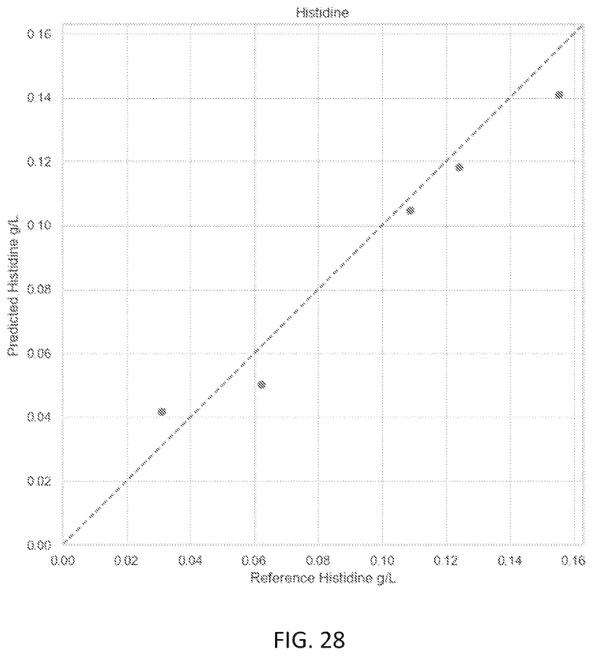

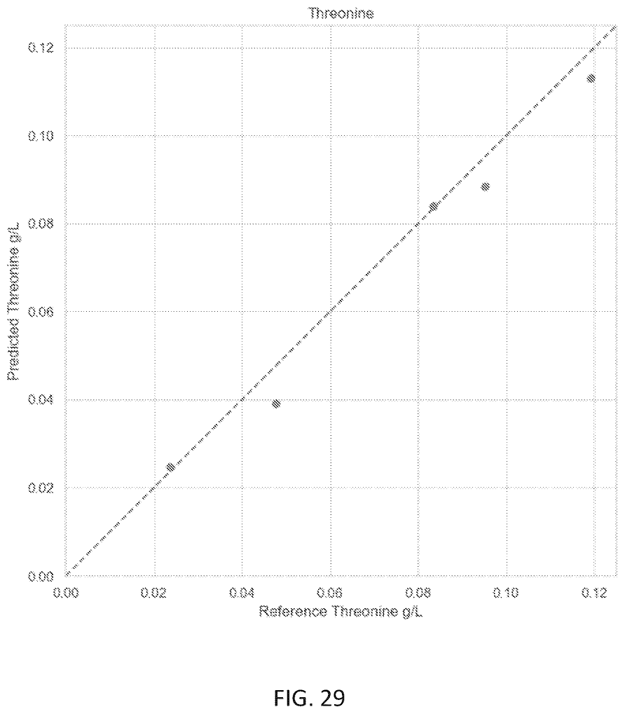

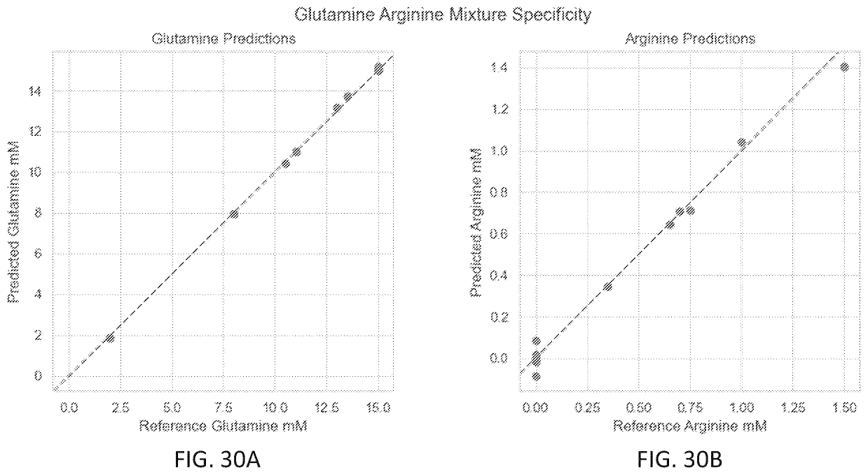

FIG. 22 shows mean R2 values, using 10-fold cross validation, for predicted concentrations of amino acids (arginine, asparagine, cysteine, glutamine, glycine, histidine, and threonine, from left to right) in water using radiofrequency signal data versus measured reference values for concentration of the same amino acids in water. FIG. 23 shows predicted values for arginine concentration in water (dotted line) using radiofrequency detection, as described herein, versus measured reference values of arginine in water (circular dots). FIG. 24 shows predicted values for glutamine concentration in water (dotted line) using radiofrequency detection, as described herein, versus measured reference values of glutamine in water (circular dots). FIG. 25 shows predicted values for asparagine concentration in water (dotted line) using radiofrequency detection, as described herein, versus measured reference values of asparagine in water (circular dots). FIG. 26 shows predicted values for glycine concentration in water (dotted line) using radiofrequency detection, as described herein, versus measured reference values of glycine in water (circular dots). FIG. 27 shows predicted values for cystine concentration in water (dotted line) using radiofrequency detection, as described herein, versus measured reference values of cystine in water (circular dots). FIG. 28 shows predicted values for histidine concentration in water (dotted line) using radiofrequency detection, as described herein, versus measured reference values of histidine in water (circular dots). FIG. 29 shows predicted values for threonine concentration in water (dotted line) using radiofrequency detection, as described herein, versus measured reference values of threonine in water (circular dots). FIG. 30A shows predicted values for glutamine concentration in a mixture of glutamine and arginine in water (dotted line) using radiofrequency detection, as described herein, versus measured reference values of glutamine in the glutamine/arginine mixture in water (circular dots). FIG. 30B shows predicted values for glutamine concentration in a mixture of arginine and arginine in water (dotted line) using radiofrequency detection, as described herein, versus measured reference values of arginine in the glutamine/arginine mixture in water (circular dots). Prediction of amino acid concentration based on radiofrequency signal analysis, as described herein, showed good correlation with measured values. Prediction of amino acid concentration was specific to amino acid species, even when measurements were made in fluids comprising a mixture of a plurality of different amino acids species.

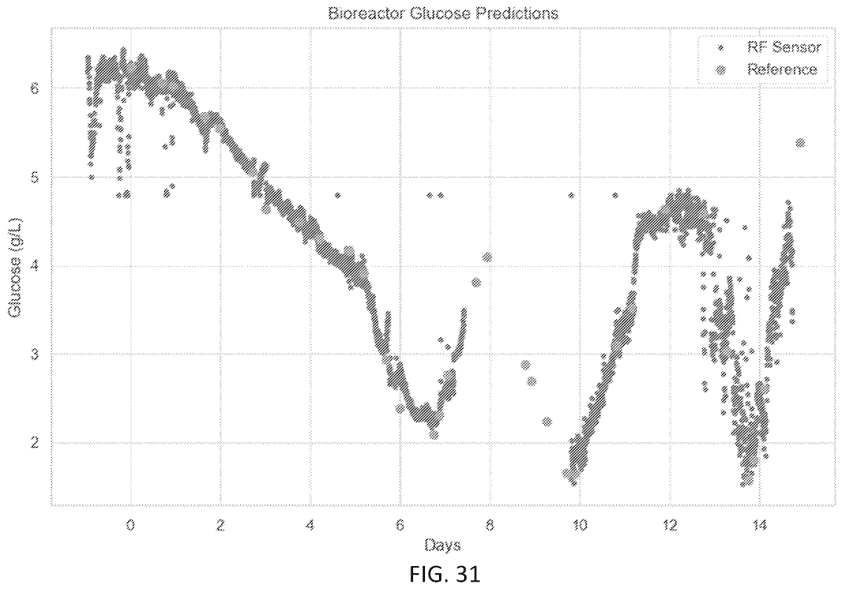

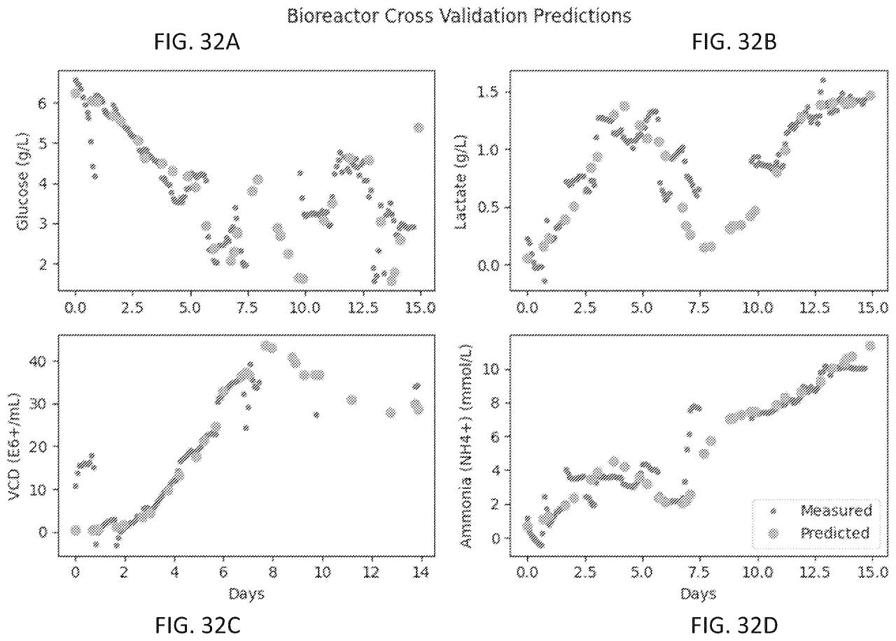

FIG. 31 shows predicted values for glucose concentration in a live bioreactor cell culture (small blue dots) using radiofrequency detection and analysis, as described herein, versus measured reference values (larger orange dots) of glucose in a live bioreactor cell culture. FIG. 32A shows predicted values for glucose concentration in a live bioreactor cell culture (small blue dots) using radiofrequency detection and analysis, as described herein, with 10-fold cross validation, versus measured reference values (larger orange dots) of glucose concentration in a live bioreactor cell culture. FIG. 32B shows predicted values for lactate concentration in a live bioreactor cell culture (small blue dots) using radiofrequency detection and analysis, as described herein, with 10-fold cross validation, versus measured reference values (larger orange dots) of lactate concentration in a live bioreactor cell culture. FIG. 32C shows predicted values for viable cell density (VCD) in a live bioreactor cell culture (small blue dots) using radiofrequency detection and analysis, as described herein, with 10-fold cross validation, versus measured reference values (larger orange dots) of VCD in a live bioreactor cell culture. FIG. 32D shows predicted values for ammonia concentration in a live bioreactor cell culture (small blue dots) using radiofrequency detection and analysis, as described herein, with 10-fold cross validation, versus measured reference values (larger orange dots) of ammonia concentration in a live bioreactor cell culture.

An analyte of interest measurable (e.g., detectable) by a system or method disclosed herein can be an amino acid (e.g., arginine, asparagine, cysteine, glutamine, glycine, histidine, or threonine), a nucleic acid, a protein, or a carbohydrate (e.g., a sugar such as glucose). In some embodiments, an analyte of interest can an ion. An analyte can be lactate. An analyte can be ammonia (e.g., ammonia dissolved in a fluid medium of a bioreactor). In some embodiments, an analyte of interest can be a potassium ion, a calcium ion, or a sodium ion.

In some embodiments, a computer system controller 150 can be configured to compare a determined (e.g., measured) concentration of an analyte of interest in a fluid medium 141 with a threshold value or tolerance value or range. In some implementations, a threshold value or tolerance value or range can be predetermined. In some implementations, a threshold value or tolerance value or range can be stored in a memory of the computer system controller 150. In some implementations, a tolerance range can be within 1%, within 5%, within 10%, within 15%, within 20%, or within 25% of a specified target concentration. In some cases, a target concentration can be from 0.01 grams per liter to 100 grams per liter, from 1 gram per liter to 100 grams per liter, or from 0.01 grams per liter to 10 grams per liter. In some embodiments, a computer system controller 150 can produce, send, and/or display a notification (e.g., to a user, for example, via a display screen and/or by sending an email or mobile phone notification) reflecting the presence or absence of one or more analytes of interest in a fluid medium 141 and/or a determined (e.g., measured) concentration of the one or more analytes in the fluid medium 141. In some implementations, a computer system controller 150 can initiate or terminate the operation of one or more components of the radiofrequency bioreactors sensor system 100 based on a determined concentration of one or more analytes in the fluid medium 141 or on the presence or absence of one or more analytes in the fluid medium.

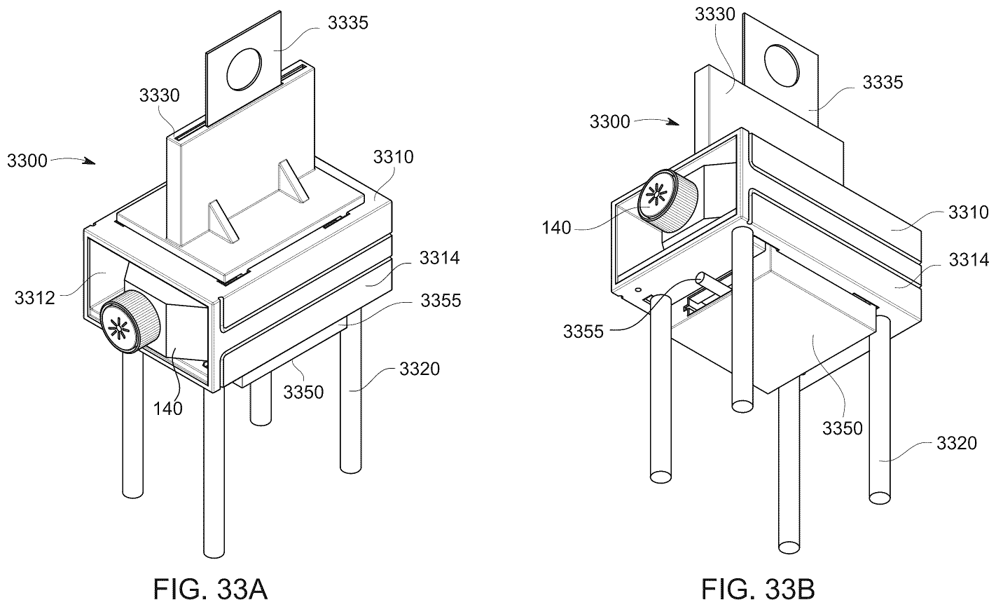

As shown in FIGS. 33A-33B, radiofrequency sensor system or RF sensor system 3300 comprises a housing enclosure 3310 configured to receive a bioprocess container 140 containing fluid 141. For securely positioning the RF system 3300 on a work platform, the housing enclosure 3310 can be supported on four support legs 3320. The housing enclosure 3310 can have a top opening and a bottom opening (not seen in FIG. 33A, and FIG. 33B), and a cavity 3312 formed by opposing side walls 3314. The cavity 3312 can be configured to receive the bioprocess container 140. Further, a top radio frequency antennae holder or top RF antenna holder 3330, including a top radio frequency antenna or top RF antenna 3335, can be adhered to, coupled to, or disposed as an integrated part of a housing enclosure 3310, around the top opening. Likewise, a bottom radio frequency antennae holder or bottom RF antenna holder 3350, including a bottom radio frequency antenna or bottom RF antenna 3355, can be adhered to, coupled to, or disposed as an integrated part of a housing enclosure 3310, around the bottom opening. It can be noted that in this example, the top and bottom RF antenna holders 3330, 3350 are orthogonal to each other, and the top RF antenna holder 3330 is in a vertical orientation and the bottom RF antenna holder 3350 is in a horizontal configuration. In alternative configurations, the top and bottom RF antenna holders 3330, 3350 can be parallel, or the top RF antenna holder 3330 can be in a horizontal orientation, and the bottom RF antenna holder 3350 can be in a vertical configuration. The top and bottom RF antennae 3335, 3355 are examples of RF transmitter and receiver antennae 112, 114, respectively, described above. Each of the top and bottom RF antennae 3335, 3355 can function as a transmitter or receiver of radiofrequency signals. For example, the top RF antenna 3335 can transmit a radio frequency signal, which passes through the opening in the top RF antenna holder 3330 and passes through the fluid 141 in the bioprocess container 140, and then passes through the opening in the bottom RF antenna holder 3350, and is received by the bottom RF antenna 3355. Further the top and bottom RF antennae 3335, 3355 can be in electronic communication with a computer processor/controller 150 (e.g., a vector network analyzer (VNA)) as described above to process the signals for analyte detection.

Optionally, housing enclosure 3310 can comprise means for securing at least a portion of bioprocess container 140 in housing enclosure 3310, such as by a covering or door to fully or partially close an opening to cavity 3312 and/or a clamp or a spring within the cavity 3312 and biased against the bioprocess container 140, or rubber pad inside of the cavity to increase friction of between the bioprocess container 140 and the housing enclosure 3310. All or a portion of housing enclosure 3310 (e.g., a side wall 3314 of housing enclosure 3310) can comprise a material or structure for electromagnetically isolating all or a portion of bioprocess container 140 or cavity 3312 from outside electromagnetic signals. For instance, all or a portion of housing enclosure 3310 can comprise a metal material or metal mesh for attenuating or blocking electromagnetic signals not originating from within cavity 3312.

In various embodiments, top and bottom radiofrequency antennae 3335 and 3355 can comprise ultrawideband antennas for various RF applications across low GHz ranging less than 10 GHz and/or a Vivaldi antenna, also known as a tapered slot antenna (TSA), which is a type of linear-polarized planar antenna. As mentioned in the above embodiments of this disclosure, top and bottom radiofrequency antennae 3335, 3355 can be configured to analyze the analyte in fluid contained in the bioprocess container 140.

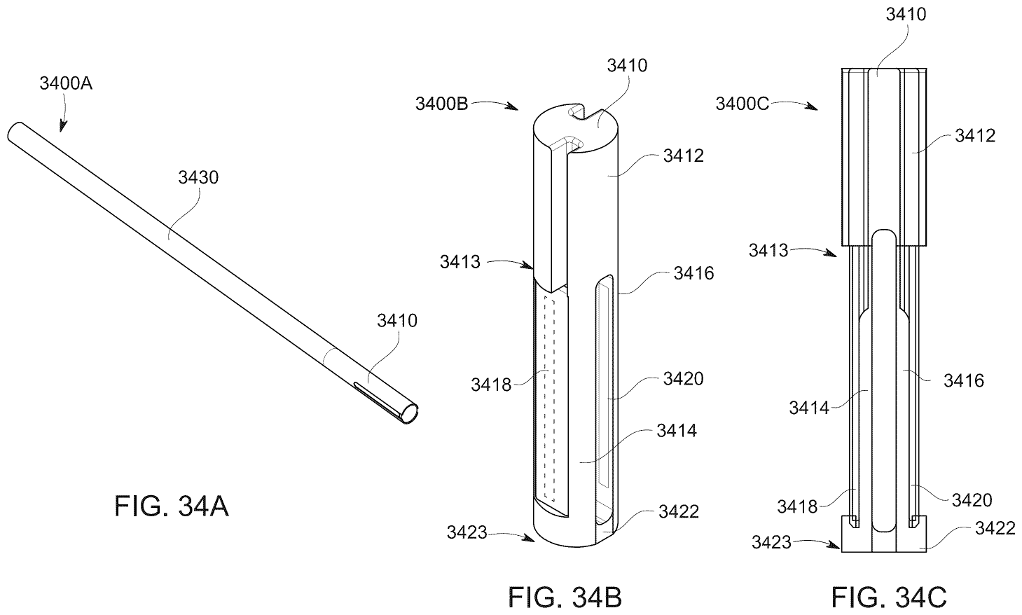

As shown in FIGS. 34A, radiofrequency sensor system 3400A comprises a probe 3410 coupled to a probe holder 3430. FIGS. 34B and 34C illustrate a perspective and cross-sectional view, respectively, of an isolated probe 3410. Radiofrequency sensor system 3400A is an example of radiofrequency system described above and can be configured to analyze, measure, detect, quantify, identify an analyte in a fluid 141 contained in a bioprocess container 140. In some implementations, the probe holder 3430 can be coupled to a port (e.g., a one-inch probe) of the bioreactor 140 such that the probe 3410 is in contact with the fluid 141 in the bioprocess container 140.

Referring to FIGS. 34B and 34C, in various embodiments, the probe 3410 can comprise one or more radiofrequency transmitter or receiver arms 3414, 3416 configured to receive a radiofrequency transmitter antenna or RF transmitter antenna 3418, or a radiofrequency receiver antenna or RF receiver antenna 3420. The RF transmitter antennae and receiver antenna 3418, 3420 are examples of RF transmitter and receiver antennae 112, 114, respectively, described above. Each of the RF transmitter antenna 3418 and/or an RF receiver antenna 3420 can be configured to receive or transmit radio frequency signals. For example, FIGS. 34A, 34B shows the probe 3410 comprising an RF transmitter arm 3414, configured to receive RF transmitter antenna 3418 and an RF receiver arm 3416 configured to receive RF receiver antenna 3420. For example, the RF transmitter arm 3414 can be embedded with the RF transmitter antenna 3418. This configuration can assist with maintaining a position of the RF transmitter antenna 3418 along the RF transmitter arm 3414. In addition, the RF receiver arm 3416 can be embedded with the RF receiver antenna 3420. Such a configuration can assist with maintaining a position of the RF receiver antenna 3420 along the RF receiver arm 3416. Additionally, the RF transmitter arm 3414 and RF receiver arm 3416 can be coupled to adhered to, integrated to a base 3412 at a proximal end 3413 and to a cap 3422 at a distal end 3423. The base 3412 of the probe can be configured to couple with the probe holder 3430. This can allow for efficient and accurate alignment between the RF transmitter antenna 3418 and RF receiver antenna 3420. For example, the RF transmitter arm 3414 and/or the RF receiver arm 3416 can have a shape, a depth, or an angle configured to maintain the RF transmitter antenna 3418 and/or an RF receiver antenna 3420 at preferred alignment position(s). In some embodiments, a preferred alignment position can comprise parallel arrangement of a first plane in which an RF transmitter antenna 3418 is entirely contained and a second plane in which an RF receiver antenna 3420 is entirely contained, wherein a straight line connecting the center of the RF transmitter antenna 3418 and the center of the RF receiver antenna 3420 is perpendicular to both the first plane and the second plane. For example, when probe 3410 is in contact with fluid 141 in the bioprocessing container 140, a portion of the fluid can be present between the RF transmitter arm 3414 and the RF receiver arm 3416. Further, the RF transmitter antenna 3418 and an RF receiver antenna 3420 can be in electronic communication with a computer processor/controller 150 (e.g., a vector network analyzer (VNA)) as described above to process the signals for analyte detection in the fluid 141.

In various embodiments probe 3410 can maintain a position and orientation of the RF transmitter antenna 3418 relative to the RF receiver antenna 3420. In some embodiments, probe 3410 and probe holder 3430 can comprise a rigid or semi-rigid material, such as a hard plastic. In some embodiments, all or a portion of the probe 3410 and probe holder 3430 can be sterilizable (e.g., autoclavable), for example, to allow reuse. For example, all or a portion of probe 3410, including all or a portion of RF transmitter arm 3414 and/or all or a portion of RF receiver arm 3416, can be made of a radio-transparent material, such as Biomed Clear SLA. In some cases, the probe 3410 and probe holder 3430 can be made of single-use material. In some cases, the probe 3410 and probe holder 3430 can be disposable (e.g., single-use). In some cases, RF transmitter antenna 3418, RF receiver antenna 3420, and/or associated communication elements can be disposable (e.g., single-use). In some implementations, an entire assembly comprising the probe 3410 and probe holder 3430, RF transmitter antenna 3418, and RF receiver antenna 3420 can be implemented as a single-use component. In some embodiments, the RF transmitter antenna 3418 and RF receiver antenna 3420 can be releasably attachable to the body of the probe 3410. In some implementations, the RF transmitter antenna 3418 and RF receiver antenna 3420 can comprise an Ultra-Wideband RF antenna.

The many features and advantages of the disclosure are apparent from the detailed specification, and thus, it is intended by the appended claims to cover all such features and advantages of the disclosure which fall within the true spirit and scope of the disclosure. Further, since numerous modifications and variations will readily occur to those skilled in the art, it is not desired to limit the disclosure to the exact construction and operation illustrated and described, and accordingly, all suitable modifications and equivalents can be resorted to, falling within the scope of the disclosure.

1. A system for measuring an analyte comprising:
a radiofrequency sensor comprising a radiofrequency transmitter and a radiofrequency receiver, the sensor configured to transmit a radiofrequency signal from the transmitter to the receiver through a fluid comprising the analyte; and
a controller comprising a processor and a non-transitory memory in communication with the sensor, the non-transitory memory comprising instructions that, when executed, cause the processor to determine a concentration of the analyte based on a resultant signal produced by the receiver.

2. The system of claim 1, wherein determining the concentration of the analyte comprises determining a change in radiofrequency signal amplitude between the radiofrequency signal transmitted by the transmitter and the radiofrequency signal received by the receiver, at one or more transmission frequencies.

3. The system of claim 2, wherein the one or more transmission frequencies comprises a range of frequencies of from 1 Hz to 10 GHz, from 1 Hz to 9 GHz, from 1 Hz to 8 GHz, from 1 Hz to 7 GHz, from 1 Hz to 6 GHz, from 1 Hz to 5 GHZ, 1 Hz to 4 GHz, from 1 Hz to 3 GHz, from 1 Hz to 2 GHz, from 1 Hz to 1 GHz, from 25 MHz to 10 GHz, from 25 MHz to 9 GHZ, from 25 MHz to 8 GHZ, from 25 MHz to 7 GHZ, from 25 MHz to 6 GHz, from 25 MHz to 5 GHZ, 25 MHz to 4 GHz, from 25 MHz to 3 GHZ, from 25 MHz to 2 GHz, or from 25 MHz to 1 GHz.

4. The system of claim 1, wherein the controller is configured to cause the transmitter to transmit the radiofrequency signal through the fluid.

5. The system of claim 1, further comprising:
a bioprocess container comprising the fluid, wherein the radiofrequency transmitter in direct contact with a first portion of the bioprocess container, and the radiofrequency receiver is in direct contact with a second portion of the bioprocess container.

6. The system of claim 5, wherein the radiofrequency transmitter is directly coupled to the first portion of the bioprocess container using an adhesive or a fastener.

7. The system of claim 5, wherein the radiofrequency receiver is directly coupled to the second portion of the bioprocess container using an adhesive or a fastener.

8. The system of claim 5, wherein the first portion of the bioprocess container directly opposes the second portion of the bioprocess container.

9. The system of claim 5, wherein the first portion of the bioprocess container is a first wall of the bioprocess container.

10. The system of claim 5, wherein the second portion of the bioprocess container is a second wall of the bioprocess container.

11. The system of claim 5, wherein the first portion of the bioprocess container and the second portion of the bioprocess container are separate portions of one wall of the bioprocess container.

12. The system of claim 1, further comprising:
a bioprocess container comprising the fluid, wherein the radiofrequency transmitter and the radiofrequency receiver are not in direct contact with the bioprocess container.

13. The system of claim 12, further comprising a housing having one or more cavities defined at least in part by one or more walls of the housing.

14. The system of claim 13, wherein the one or more cavities are shaped and sized to receive at least a portion of the bioprocess container.

15. The system of claim 12, wherein the radiofrequency transmitter is directly coupled to a first wall of the one or more walls.

16-49. (canceled)

50. A method for measuring an analyte, the method comprising:
transmitting a radiofrequency signal from a radiofrequency transmitter into a fluid comprising an analyte;
receiving a resultant radiofrequency signal at a radiofrequency receiver, wherein the resultant radiofrequency signal is the transmitted radiofrequency signal after it has passed through at least a portion of fluid; and
analyzing the radiofrequency signal received by the receiver to determine a concentration of the analyte in the fluid.

51. The method of claim 50, wherein the fluid is comprised in a bioprocess container and wherein the radiofrequency transmitter in direct contact with a first portion of the bioprocess container and the radiofrequency receiver is in direct contact with a second portion of the bioprocess container.

52. The method of claim 51, wherein the radiofrequency transmitter is directly coupled to the first portion of the bioprocess container using an adhesive or a fastener.

53. The method of claim 51, wherein the radiofrequency receiver is directly coupled to the second portion of the bioprocess container using an adhesive or a fastener.

54. The method of claim 50, wherein the first portion of the bioprocess container directly opposes the second portion of the bioprocess container.

55-126. (canceled)
